(19) United States
(12) Patent Application Publication
(10) Pub. No.: US 2026/0055055 A1
(43) Pub. Date: Feb. 26, 2026

(54) PROCESS FOR MAKING SELADELPAR

(71) Applicant: CymaBay Therapeutics, Inc., Foster City, CA (US)

(72) Inventors: Xin CHEN, San Ramon, CA (US); Bharat GURALE, Riverview, MI (US); Dumitru IONESCU, Ann Arbor, MI (US); Eric Jason KISER, Saline, MI (US); Stephen STEFFKE, Weidman, MI (US); Hi TAING, Riverview, MI (US); Buchi VADDULA, Riverview, MI (US); Fei YU, Wilmington, MA (US)

(21) Appl. No.: 19/285,934

(22) Filed: Jul. 30, 2025

Related U.S. Application Data
(60) Provisional application No. 63/677,935, filed on Jul. 31, 2024.

(51) Int. Cl. C07C 323/62; C07C 229/26; C07C 319/20

(52) U.S. Cl. CPC C07C 323/62; C07C 229/26; C07C 319/20

(57) ABSTRACT

The present disclosure relates generally to processes for preparing seladelpar, an agonist of peroxisome proliferator-activated receptor (PPAR)-delta, and compositions thereof.

### CROSS-REFERENCE TO RELATED APPLICATIONS

This application claims the benefit to U.S. Provisional Application No. 63/677,935, filed Jul. 31, 2024, which is hereby incorporated by reference in its entirety.

### FIELD

The present disclosure relates generally to processes for preparing seladelpar, an agonist of peroxisome proliferator-activated receptor (PPAR)-delta (PPARδ), and compositions thereof.

### BACKGROUND

Primary biliary cholangitis (PBC) is a liver disease that is characterized by the destruction of small intrahepatic bile ducts and accumulation of toxic bile acids. This results in cholestasis, inflammation, and biliary fibrosis, which can lead to cirrhosis and liver failure. PPAR-delta has broad expression in hepatocytes, cholangiocytes, Kupper cells, and stellate cells, each of which play a key role in the pathobiology of PBC.

Seladelpar selectively activates PPAR-delta and is thus useful for the treatment of PBC. However, there remains a need for improved or alternate processes to prepare seladelpar.

### SUMMARY

Provided herein are methods of making seladelpar, i.e. a compound of formula I, and compositions thereof. Some embodiments provide for a method of making a compound of formula I:

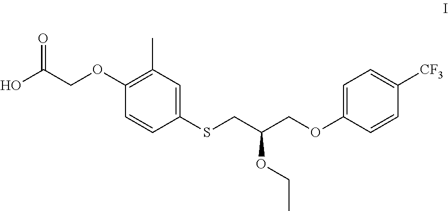 [SMILES: CCO[C@H](COc1ccc(C(F)(F)F)cc1)CSc1ccc(OCC(=O)O)c(C)c1]

or a pharmaceutically acceptable salt or hydrate thereof, comprising:
contacting a compound of formula (A):

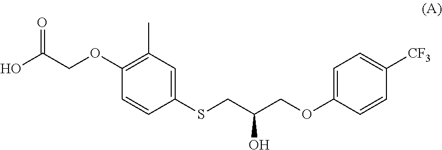 [SMILES: Cc1cc(SC[C@H](O)COc2ccc(C(F)(F)F)cc2)ccc1OCC(=O)O]

or a salt thereof, with CH₃CH₂-LG, wherein LG is a leaving group, under conditions sufficient to form a compound of formula I or a pharmaceutically acceptable salt or hydrate thereof.

Some embodiments provide for a method of making a compound of formula I:

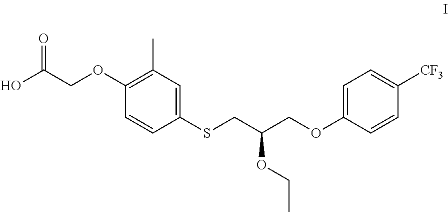 [SMILES: CCO[C@H](COc1ccc(C(F)(F)F)cc1)CSc1ccc(OCC(=O)O)c(C)c1]

or a pharmaceutically acceptable salt or hydrate thereof, comprising:
(a) contacting a compound of formula (B):

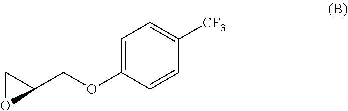 [SMILES: FC(F)(F)c1ccc(OC[C@@H]2CO2)cc1]

with a compound of formula (C):

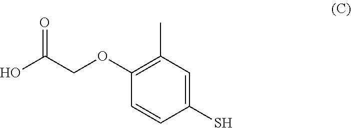

or a salt thereof, under conditions sufficient to form a compound of formula (A):

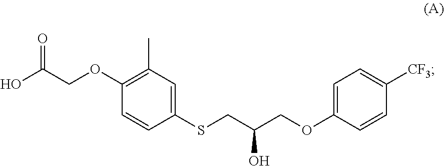 [SMILES: Cc1cc(SC[C@H](O)COc2ccc(C(F)(F)F)cc2)ccc1OCC(=O)O]

or a salt thereof;
and (b) contacting a compound of formula (A), or a salt thereof, with CH₃CH₂-LG, wherein LG is a leaving group, under conditions sufficient to form a compound of formula I or a pharmaceutically acceptable salt or hydrate thereof.

Also provided herein is a method of making a compound of formula I:

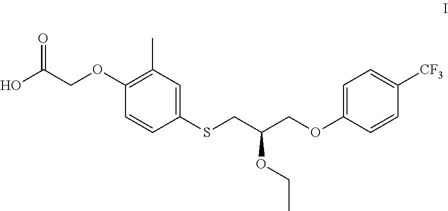 [SMILES: CCO[C@H](COc1ccc(C(F)(F)F)cc1)CSc1ccc(OCC(=O)O)c(C)c1]

or a pharmaceutically acceptable salt or hydrate thereof, comprising:
(a) contacting a compound of formula (G):

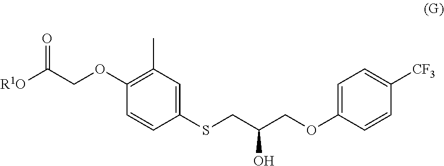

wherein R¹ is C₁₋₆ alkyl, C₂₋₆ alkenyl, C₂₋₆ alkynyl, C₃₋₁₀ cycloalkyl, heterocyclyl, aryl, or heteroaryl; with CH₃CH₂-LG, wherein LG is a leaving group, in the presence of sodium hydroxide and dimethylsulfoxide (DMSO) and water, under conditions sufficient to form a compound of formula II:

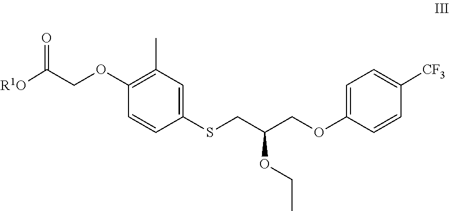

and (b) hydrolyzing the compound of formula III under conditions sufficient to form a compound of formula I or a pharmaceutically acceptable salt or hydrate thereof.

Some embodiments provide for a composition comprising a lysine salt of a compound of formula I-a:

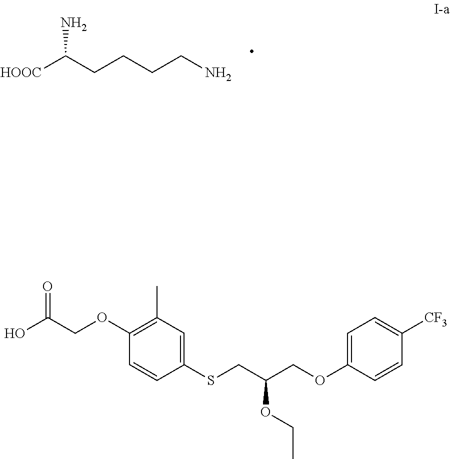 [SMILES: NCCCC[C@@H](N)C(=O)O ; CCO[C@H](COc1ccc(C(F)(F)F)cc1)CSc1ccc(OCC(=O)O)c(C)c1]

wherein the composition comprises less than about 2% a/a of a compound selected from:

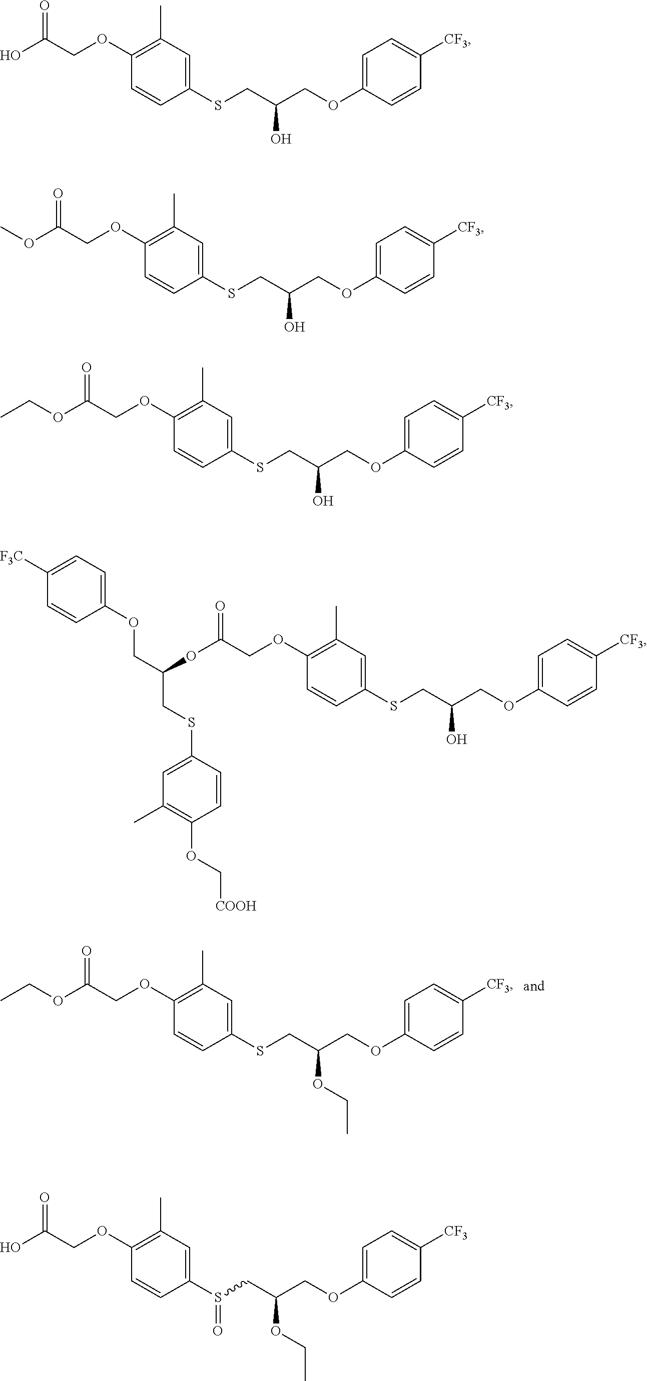 [SMILES: Cc1cc(SC[C@H](O)COc2ccc(C(F)(F)F)cc2)ccc1OCC(=O)O ; COC(=O)COc1ccc(SC[C@H](O)COc2ccc(C(F)(F)F)cc2)cc1C ; CCOC(=O)COc1ccc(SC[C@H](O)COc2ccc(C(F)(F)F)cc2)cc1C ; Cc1cc(SC[C@@H](COc2ccc(C(F)(F)F)cc2)OC(=O)COc2ccc(SC[C@H](O)COc3ccc(C(F)(F)F)cc3)cc2C)ccc1OCC(=O)O ; CCOC(=O)COc1ccc(SC[C@@H](COc2ccc(C(F)(F)F)cc2)OCC)cc1C ; CCO[C@H](COc1ccc(C(F)(F)F)cc1)CS(=O)c1ccc(OCC(=O)O)c(C)c1]

or a combination thereof.

Some embodiments provide for a composition comprising a compound of formula I:

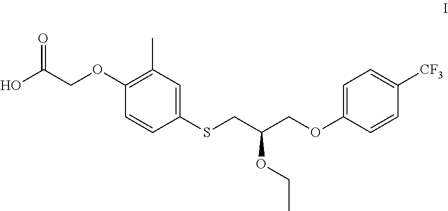 [SMILES: CCO[C@H](COc1ccc(C(F)(F)F)cc1)CSc1ccc(OCC(=O)O)c(C)c1]

or a pharmaceutically acceptable salt or hydrate thereof, wherein the composition comprises less than 0.1% a/a of a compound of formula II:

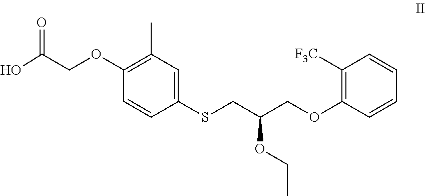 [SMILES: CCO[C@H](COc1ccccc1C(F)(F)F)CSc1ccc(OCC(=O)O)c(C)c1]

or a pharmaceutically acceptable salt thereof.

Some embodiments provide for a composition comprising a compound of formula I-b1:

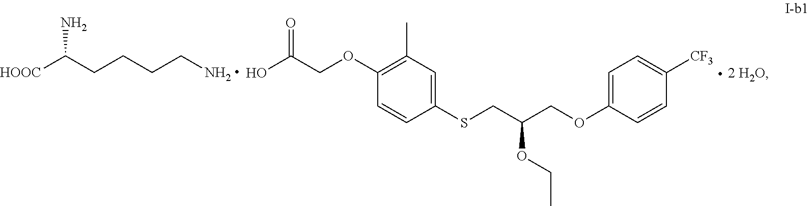 [SMILES: NCCCC[C@@H](N)C(=O)O ; CCO[C@H](COc1ccc(C(F)(F)F)cc1)CSc1ccc(OCC(=O)O)c(C)c1]

wherein the composition comprises less than 0.1% a/a of a compound of formula II-a:

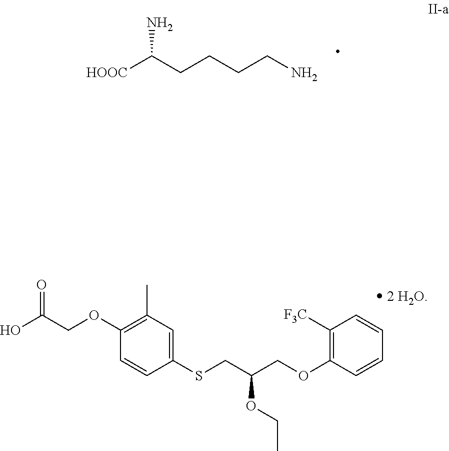 [SMILES: NCCCC[C@@H](N)C(=O)O ; CCO[C@H](COc1ccccc1C(F)(F)F)CSc1ccc(OCC(=O)O)c(C)c1]

Some embodiments provide for a composition comprising a compound of formula I-b1:

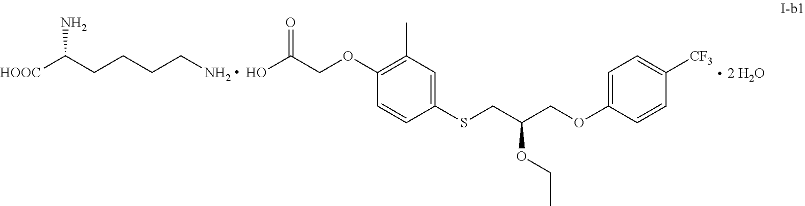 [SMILES: NCCCC[C@@H](N)C(=O)O ; CCO[C@H](COc1ccc(C(F)(F)F)cc1)CSc1ccc(OCC(=O)O)c(C)c1]

wherein the composition comprises at least about 97% w/w of the compound of formula I-b1, and less than about 2% w/w of a compound selected from:

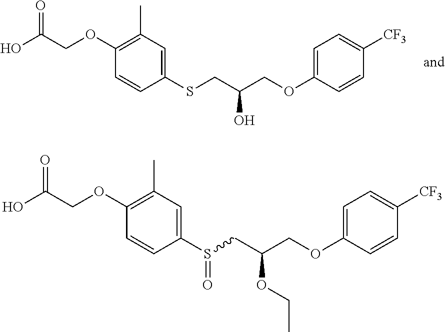 [SMILES: Cc1cc(SC[C@H](O)COc2ccc(C(F)(F)F)cc2)ccc1OCC(=O)O ; CCO[C@H](COc1ccc(C(F)(F)F)cc1)CS(=O)c1ccc(OCC(=O)O)c(C)c1]

or a combination thereof.

### DETAILED DESCRIPTION

### Definitions

As used in the present specification, the following words and phrases are generally intended to have the meanings as set forth below, except to the extent that the context in which they are used indicates otherwise.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±2.5%. In certain other embodiments, the term “about” includes the indicated amount±1%. Also, to the term “about X” includes description of “X”.

Recitation of numeric ranges of values throughout the disclosure is intended to serve as a shorthand notation of referring individually to each separate value falling within the range inclusive of the values defining the range, and each separate value is incorporated in the specification as it were individually recited herein.

The term “alkyl” as used herein, means a straight or branched, saturated hydrocarbon chain containing from 1 to 30 carbon atoms. The term “lower alkyl” or “C₁-C₆-alkyl” means a straight or branched chain hydrocarbon containing from 1 to 6 carbon atoms. The term “C₁-C₄-alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

“Alkenyl” refers to an alkyl group containing at least one (e.g., 1-3, or 1) carbon-carbon double bond and having from 2 to 20 carbon atoms (i.e., C₂₋₂₀ alkenyl), 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include, e.g., ethenyl, propenyl, butadienyl (including 1,2-butadienyl and 1,3-butadienyl).

“Alkynyl” refers to an alkyl group containing at least one (e.g., 1-3, or 1) carbon-carbon triple bond and having from 2 to 20 carbon atoms (i.e., C₂₋₂₀ alkynyl), 2 to 12 carbon atoms (i.e., C₂₋₁₂ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 14 ring carbon atoms (i.e., C₃₋₁₂ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl, and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁-20 heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, thiophenyl (i.e., thienyl), tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (═O) or N-oxide (—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiomorpholinyl, thiamorpholinyl, 1-oxo-thiomorpholinyl, and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

As used herein, the term “contacting” refers to the process of bringing into contact at least two distinct species such that they can react. It should be appreciated, however, that the resulting reaction product can be produced directly from a reaction between the added reagents or from an intermediate from one or more of the added reagents which can be produced in the reaction mixture.

The term “reaction conditions” is intended to refer to the physical and/or environmental conditions under which a chemical reaction proceeds. Examples of reaction conditions include, but are not limited to, one or more of following: reaction temperature, solvent, pH, pressure, reaction time, mole ratio of reactants, the presence of a base or acid, one or more protecting groups, or catalyst, radiation, etc. Reaction conditions may be named after the particular chemical reaction in which the conditions are employed, such as, coupling conditions, hydrogenation conditions, acylation conditions, reduction conditions, etc. Reaction conditions for most reactions are generally known to those skilled in the art or can be readily obtained from the literature. Exemplary reaction conditions sufficient for performing the chemical transformations provided herein can be found throughout, and in particular, the examples below. It is also contemplated that the reaction conditions can include reagents in addition to those listed in the specific reaction.

As used herein, “under conditions sufficient” is intended to refer to the reaction conditions under which the desired chemical reaction may proceed.

“Hydrolyzing” or “hydrolysis” as used herein refers to the cleavage by water of a carboxylic ester into a carboxylic acid and an alcohol. Exemplary hydrolyzing agents include, but are not limited to, inorganic bases, such as sodium carbonate, sodium bicarbonate, potassium carbonate, and potassium bicarbonate, and alkoxide bases, such as sodium methoxide and potassium methoxide.

As used herein, the verb “hydrate” (and forms thereof) or the term “hydration” refers to a chemical reaction in which water is added to a substance or compound.

The term “leaving group” refers to an atom or a group of atoms that is displaced in a chemical reaction as stable species taking with it the bonding electrons. The non-limiting examples of a leaving group include, halo, methanesulfonyloxy, p-toluenesulfonyloxy, trifluoromethanesulfonyloxy, nonafluorobutanesulfonyloxy, (4-bromo-benzene) sulfonyloxy, (4-nitro-benzene) sulfonyloxy, (2-nitro-benzene)-sulfonyloxy, (4-isopropyl-benzene) sulfonyloxy, (2,4,6-tri-isopropyl-benzene)-sulfonyloxy, (2,4,6-trimethyl-benzene) sulfonyloxy, (4-tertbutyl-benzene) sulfonyloxy, benzenesulfonyloxy, (4-methoxy-benzene) sulfonyloxy, and the like.

The term “reducing agent” refers to an element or compound that loses an electron to an oxidizing agent in a redox reaction. Reducing agents increase the electron density on carbon centers, either by bond formation between the carbon and a less electronegative atom, or by bond breaking between the carbon and a more electronegative atom. Reducing agents usually accomplish this change in electron density by the addition of hydrogen, or the substitution of hydrogen for an electronegative atom on the carbon center.

As used herein, the term “salt” refers to a compound formed by the reaction of an acid and a base, resulting in the formation of a positively charged cation and a negatively charged anion. In general, a salt is defined as a compound that is formed by the combination of positively and negatively charged ions, where the charges of the ions result in a neutral compound. Salts can be either inorganic or organic. As used herein, the term “salt” includes partially or fully ionized salt forms. In some embodiments, the salt is fully ionized.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables. The term “pharmaceutically acceptable salt” of a given compound refers to salts that retain the biological effectiveness and properties of the given compound, and which are not biologically or otherwise undesirable. Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines. Specific examples of suitable amines include, by way of example only, isopropyl amine, trimethyl amine, diethyl amine, tri (iso-propyl) amine, tri (n-propyl) amine, ethanolamine, diethanolamine, 2-dimethylamino ethanol, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The term “solvate” refers to a complex formed by combination of solvent molecules with molecules or ions of the solute. The solvent can be an organic compound, an inorganic compound, or a mixture of both. As used herein, the term “solvate” includes a “hydrate” (i.e., a complex formed by combination of water molecules with molecules or ions of the solute), hemi-hydrate, channel hydrate, etc. Some examples of solvents include, but are not limited to, acetonitrile, methanol, N,N-dimethylformamide, tetrahydrofuran, 2-methyltetrahydrofuran, dimethylsulfoxide, and water. In general, the solvated forms are equivalent to unsolvated forms and are encompassed within the scope of the present disclosure.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

As used herein, “% w/w” refers to the weight of a component based on the total weight of a composition comprising the component. For instance, if component 1 is present in an amount of 50% in a 100 mg composition, component 1 is present in an amount of 50 mg.

As used herein, “% a/a” refers to the area of a component based on the total area of all components in a composition and may be calculated according to methods known in the art. In some embodiments, % a/a may be determined by measuring the area(s) of peak(s) (e.g., as measured by HPLC or according to other methods described herein or known in the art) and calculating accordingly.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Subject” refers to an animal, such as a mammal (including a human), that has been or will be the object of treatment, observation, or experiment. The methods described herein may be useful in human therapy and/or veterinary applications. In some embodiments, the subject is a mammal. In one embodiment, the subject is a human.

The term “therapeutically effective amount” or “effective amount” of a compound or composition described herein means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of a liver disease, such as primary biliary cholangitis (PBC). The therapeutically effective amount may vary depending on the subject, and disease or condition being treated, the weight and age of the subject, the severity of the disease or condition, and the manner of administering, which can readily be determined by one or ordinary skill in the art.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are embraced by the present disclosure and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also embraced by the present disclosure and are disclosed herein just as if each and every such sub-combination was individually and explicitly disclosed herein.

### Processes

The present disclosure relates to processes for providing seladelpar, i.e. a compound of formula I, or a salt, or pharmaceutically acceptable salt, or hydrate, thereof.

It is contemplated that the processes described herein may achieve seladelpar, or a salt thereof, or a pharmaceutically acceptable salt thereof, or hydrate thereof, or intermediates described herein, or compositions of each thereof, in large-scale and/or high purity.

Provided herein is a method of making a compound of formula I:

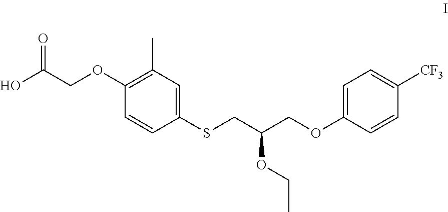 [SMILES: CCO[C@H](COc1ccc(C(F)(F)F)cc1)CSc1ccc(OCC(=O)O)c(C)c1]

or a pharmaceutically acceptable salt or hydrate thereof, comprising:
contacting a compound of formula (A):

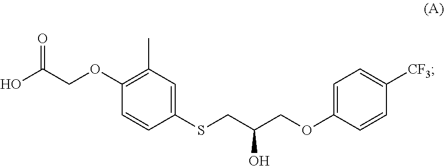 [SMILES: Cc1cc(SC[C@H](O)COc2ccc(C(F)(F)F)cc2)ccc1OCC(=O)O]

or a salt thereof, with CH₃CH₂-LG, wherein LG is a leaving group, under conditions sufficient to form a compound of formula I or a pharmaceutically acceptable salt or hydrate thereof.

In some embodiments, methods described herein comprise contacting a compound of formula (A), or a salt thereof, with CH₃CH₂-LG in the presence of sodium hydroxide and dimethylsulfoxide (DMSO) and water.

Some embodiments provide for a method of making a compound of formula I, or a pharmaceutically acceptable salt or hydrate thereof, comprising:
contacting a compound of formula (A), or a salt thereof, with CH₃CH₂—Br under conditions sufficient to form a compound of formula I or a pharmaceutically acceptable salt or hydrate thereof.

Some embodiments provide for a method of making a compound of formula I, or a pharmaceutically acceptable salt or hydrate thereof, comprising:
contacting a compound of formula (A), or a salt thereof, with CH₃CH₂—Br in the presence of sodium hydroxide and dimethylsulfoxide (DMSO) and water under conditions sufficient to form a compound of formula I or a pharmaceutically acceptable salt or hydrate thereof.

In some embodiments, contacting a compound of formula (A), or a salt thereof, with CH₃CH₂-LG (such as CH₃CH₂—Br) is at ambient temperature. In some embodiments, sodium hydroxide is added after addition of CH₃CH₂-LG (such as CH₃CH₂—Br).

In some embodiments, methods described herein comprise about 2 to about 3 equivalents of sodium hydroxide, about 5 to 7 volumes of DMSO, and about 1 to 2 equivalents of CH₃CH₂—Br. In some embodiments, methods described herein comprise about 2.5 to about 3 equivalents of sodium hydroxide, about 5 to 7 volumes of DMSO, and about 1 to 2 equivalents of CH₃CH₂—Br. In some embodiments, the temperature is about 10° C. to about 30° C. In some embodiments, the temperature is about 15° C. to about 25° C.

Some embodiments provide for a method of making a compound of formula I:

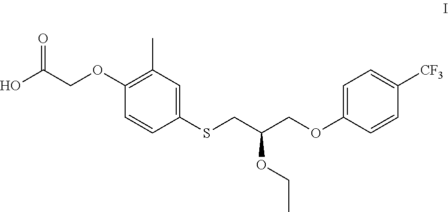 [SMILES: CCO[C@H](COc1ccc(C(F)(F)F)cc1)CSc1ccc(OCC(=O)O)c(C)c1]

or a pharmaceutically acceptable salt or hydrate thereof, comprising:
(a) contacting a compound of formula (B):

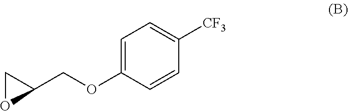 [SMILES: FC(F)(F)c1ccc(OC[C@@H]2CO2)cc1]

with a compound of formula (C):

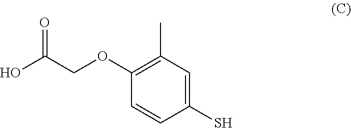

or a salt thereof,
under conditions sufficient to form a compound of formula (A):

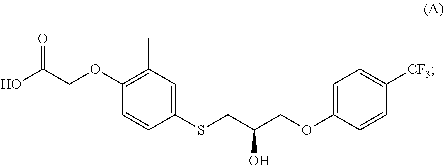 [SMILES: Cc1cc(SC[C@H](O)COc2ccc(C(F)(F)F)cc2)ccc1OCC(=O)O]

or a salt thereof;
and (b) contacting a compound of formula (A), or a salt thereof, with CH₃CH₂-LG, wherein LG is a leaving group,
under conditions sufficient to form a compound of formula I or a pharmaceutically acceptable salt or hydrate thereof.

In some embodiments, LG is a leaving group such that CH₃CH₂-LG is a sufficient ethylating agent (i.e. an agent capable of adding an ethyl group to a compound). In some embodiments, LG is a halo, triflate, tosylate, or mesylate. In some embodiments, LG is halo. In some embodiments, LG is Br or Cl. In some embodiments, LG is Br.

In some embodiments, step (a) comprises a base. In some embodiments, the base is an inorganic base. In some embodiments, the base is an organic base. In some embodiments, the base is triethylamine.

In some embodiments, step (a) comprises a solvent. In some embodiments, the solvent of step (a) is an organic solvent. In some embodiments, In some embodiments, the solvent of step (a) is an organic, aprotic solvent. In some embodiments, the solvent of step (a) is dimethylformamide (DMF). In some embodiments, the solvent is present in an amount of about 2 to about 3.5 volumes.

In some embodiments, the temperature of step (a) is about 25° C. to about 35° C. In some embodiments, the temperature of step (a) is about 30° C.

In some embodiments, the base of step (a) is triethylamine, and the solvent is DMF.

In some embodiments, step (b) comprises a base and a solvent. In some embodiments, the base of step (b) is an organic base. In some embodiments, the base of step (b) is an inorganic base. In some embodiments, the base is sodium hydroxide. In some embodiments, the sodium hydroxide is in an aqueous solution. In some embodiments, the solvent of step (b) is an organic solvent and water. In some embodiments, the solvent of step (b) is an organic, aprotic solvent and water. In some embodiments, the solvent of step (b) is dimethylsulfoxide (DMSO). In some embodiments, the solvent of step (b) is DMSO and water.

In some embodiments, the base (of step (b)) is sodium hydroxide, and the solvent is DMSO and water.

In some embodiments, sodium hydroxide is added after addition of CH₃CH₂-LG (such as CH₃CH₂—Br).

In some embodiments, LG is Br, and step (b) comprises about 2 to about 3 equivalents of sodium hydroxide, about 5 to 7 volumes of DMSO, and about 1 to 2 equivalents of CH₃CH₂—Br. In some embodiments, LG is Br, and step (b) comprises about 2.5 to about 3 equivalents of sodium hydroxide, about 5 to 7 volumes of DMSO, and about 1 to 2 equivalents of CH₃CH₂—Br.

In some embodiments, step (b) is at ambient temperature. In some embodiments, the temperature of step (b) is about 10° C. to about 30° C. In some embodiments, the temperature of step (b) is about 15° C. to about 25° C.

Some embodiments provide for a method of making a compound of formula I:

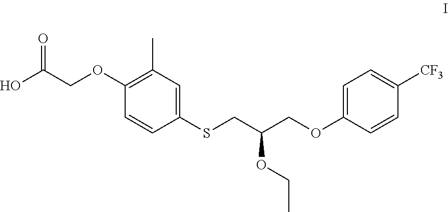 [SMILES: CCO[C@H](COc1ccc(C(F)(F)F)cc1)CSc1ccc(OCC(=O)O)c(C)c1]

or a pharmaceutically acceptable salt or hydrate thereof,
comprising:
(a) contacting a compound of formula (B):

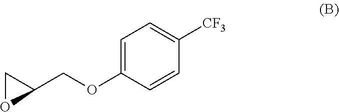 [SMILES: FC(F)(F)c1ccc(OC[C@@H]2CO2)cc1]

with a compound of formula (C):

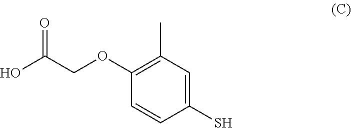

or a salt thereof,
in the presence of triethylamine and DMF under conditions sufficient to form a compound of formula (A):

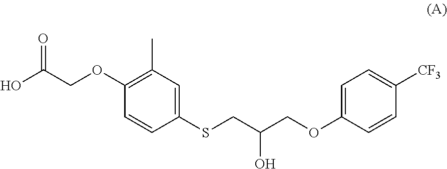 [SMILES: Cc1cc(SCC(O)COc2ccc(C(F)(F)F)cc2)ccc1OCC(=O)O]

or a salt thereof;
and (b) contacting a compound of formula (A), or a salt thereof, with CH₃CH₂—Br in the presence of sodium hydroxide and DMSO and water under conditions sufficient to form a compound of formula I or a pharmaceutically acceptable salt or hydrate thereof.

Some embodiments of the methods described herein further comprise:
contacting a compound of formula (D):

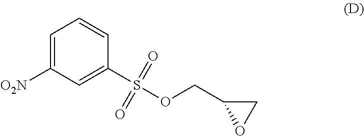 [SMILES: O=[N+]([O-])c1cccc(S(=O)(=O)OC[C@@H]2CO2)c1]

with a compound of formula (E):

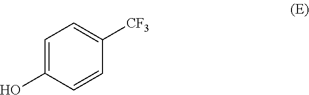 [SMILES: Oc1ccc(C(F)(F)F)cc1]

in the presence of a base under conditions sufficient to form a compound of formula (B).

In some embodiments, the base is an inorganic base. In some embodiments, the base is cesium carbonate, potassium carbonate, or sodium carbonate. In some embodiments, the base is cesium carbonate or potassium carbonate. In some embodiments, the base is potassium carbonate. In some embodiments, the base is cesium carbonate. In some embodiments, the base is present in an amount of about 0.5 equivalent to about 2.0 equivalents, and the compound of formula (D) in an amount of about 1 equivalent. In some embodiments, the base is present in an amount of about 1 equivalent to about 2 equivalents. In some embodiments, the base is present in an amount of about 1.5 equivalents.

In some embodiments, the compound of formula (D) is present in an amount of about 1 equivalent, and the compound of formula (E) is present in an amount of about 1 equivalent.

In some embodiments, contacting a compound of formula (D) with a compound of formula (E) comprises a solvent. In some embodiments, the solvent is an organic solvent. In some embodiments, the solvent is ethyl acetate or dimethyl sulfoxide. In some embodiments, the solvent is ethyl acetate. In some embodiments, the solvent is dimethyl sulfoxide.

Some embodiments provide for contacting a compound of formula (D) with a compound of formula (E) at a temperature of about 10° C. to about 80° C. In some embodiments, the temperature is about 10° C. to about 30° C. In some embodiments, the temperature is about 15° C. to about 25° C.

Some embodiments of the methods described herein further comprise:
contacting a compound of formula (D) with a compound of formula (E) in the presence of a cesium carbonate and dimethyl sulfoxide under conditions sufficient to form a compound of formula (B).

Some embodiments of the methods described herein further comprise:
contacting a compound of formula (F):

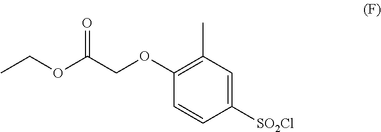 [SMILES: CCOC(=O)COc1ccc(S(=O)(=O)Cl)cc1C]

with a reducing agent under conditions sufficient to form a compound of formula (C) or a salt thereof.

In some embodiments, the reducing agent is tin powder in the presence of an acid. In some embodiments, the tin powder is present in an amount of about 3 equivalents to about 4 equivalents, and compound of formula (F) is present in about 1 equivalent. In some embodiments, the tin powder is present in an amount of about 3.5 equivalents, and compound of formula (F) is present in about 1 equivalent.

In some embodiments, the acid is acetic acid, hydrochloric acid, or a mixture thereof. In some embodiments, the acid is acetic acid and hydrochloric acid. In some embodiments, the acetic acid and hydrochloric acid are added concurrently. In some embodiments, acetic acid and hydrochloric acid are added sequentially.

In some embodiments, the acid is present in about 7 to about 10 volumes. In some embodiments, the acetic acid and hydrochloric acid are present in about 4 volumes each.

Some embodiments provide for contacting a compound of formula (F) with a reducing agent at a temperature of about 50° C. to about 70° C. In some embodiments, the temperature is about 55° C. to about 65° C.

Some embodiments of the methods described herein further comprise:
contacting a compound of formula (F) with tin powder in the presence of an acid under conditions sufficient to form a compound of formula (C) or a salt thereof, wherein the acid is acetic acid, hydrochloric acid, or a mixture thereof.

Some embodiments provide for a method of making a compound of formula I:

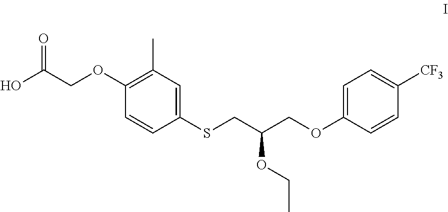 [SMILES: CCO[C@H](COc1ccc(C(F)(F)F)cc1)CSc1ccc(OCC(=O)O)c(C)c1]

or a pharmaceutically acceptable salt or hydrate thereof,
comprising:
(a) contacting a compound of formula (G):

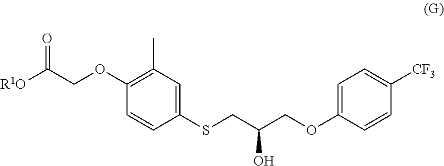

wherein R¹ is C₁₋₆ alkyl, C₂₋₆ alkenyl, C₂₋₆ alkynyl, C₃₋₁₀ cycloalkyl, heterocyclyl, aryl, or heteroaryl;
with CH₃CH₂-LG, wherein LG is a leaving group, in the presence of sodium hydroxide
under conditions sufficient to form a compound of formula III:

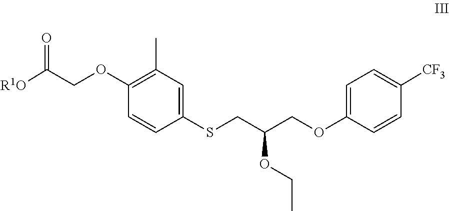

and (b) hydrolyzing the compound of formula III under conditions sufficient to form a compound of formula I or a pharmaceutically acceptable salt or hydrate thereof.

Some embodiments provide for a method of making a compound of formula I:

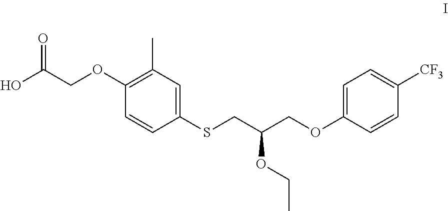 [SMILES: CCO[C@H](COc1ccc(C(F)(F)F)cc1)CSc1ccc(OCC(=O)O)c(C)c1]

or a pharmaceutically acceptable salt or hydrate thereof,
comprising:
(a) contacting a compound of formula (G):

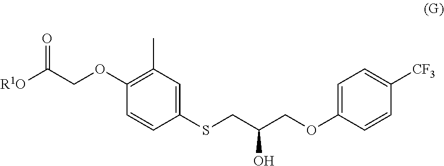

wherein R¹ is C₁₋₆ alkyl, C₂₋₆ alkenyl, C₂₋₆ alkynyl, C₃₋₁₀ cycloalkyl, heterocyclyl, aryl, or heteroaryl;
with CH₃CH₂-LG, wherein LG is a leaving group,
in the presence of sodium hydroxide and dimethylsulfoxide (DMSO) and water,
under conditions sufficient to form a compound of formula III:

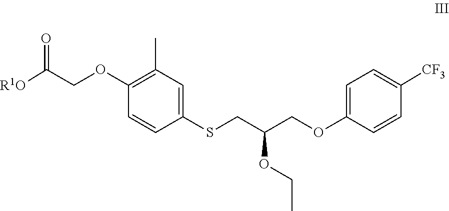

and (b) hydrolyzing the compound of formula III under conditions sufficient to form a compound of formula I or a pharmaceutically acceptable salt or hydrate thereof.

In some embodiments, LG is Br. In some embodiments, R¹ is C₁₄ alkyl. In some embodiments, R¹ is methyl or ethyl. In some embodiments, LG is Br, and R¹ is C₁₄ alkyl.

Some embodiments of the methods described herein further comprise:
contacting a compound of formula I with L-lysine under conditions sufficient to form a lysine salt of a compound of formula I-a1:

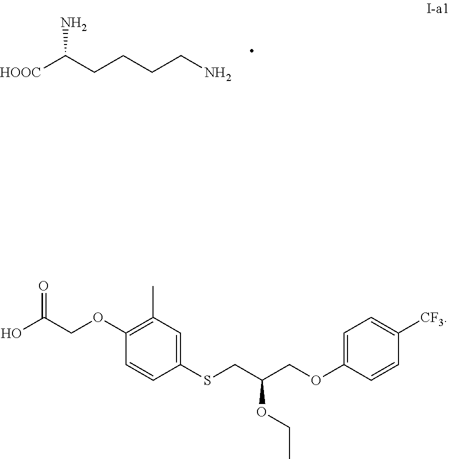 [SMILES: NCCCC[C@@H](N)C(=O)O ; CCO[C@H](COc1ccc(C(F)(F)F)cc1)CSc1ccc(OCC(=O)O)c(C)c1]

In some embodiments, L-lysine is L-lysine hydrate. In some embodiments, L-lysine hydrate is present in an amount of about 1 equivalent.

In some embodiments, contacting a compound of formula I with L-lysine comprises a solvent. In some embodiments, the solvent is water, an alcohol, or a combination thereof. In some embodiments, the solvent is water, ethanol, methanol, or a combination thereof. In some embodiments, the solvent is a combination of water, ethanol, and methanol. In some embodiments, the amount of ethanol is about 10 to 20 volumes, the amount of methanol is about 2 to 5 volumes, and the amount of water is about less than about 0.5 volumes. In some embodiments, the amount of ethanol is about 15 volumes, the amount of methanol is about 4 volumes, and the amount of water is about 0.2 volumes. In some embodiments, the amount of ethanol is about 8 volumes, and the amount of methanol is about 2 volumes.

In some embodiments, the solvent is refluxed and then cooled to room temperature.

Some embodiments of the methods described herein further comprise contacting the lysine salt of a compound of formula I-a1 with methanol, ethanol, water, or a mixture thereof, under conditions sufficient to form a recrystallized lysine salt of a compound of formula I-a2:

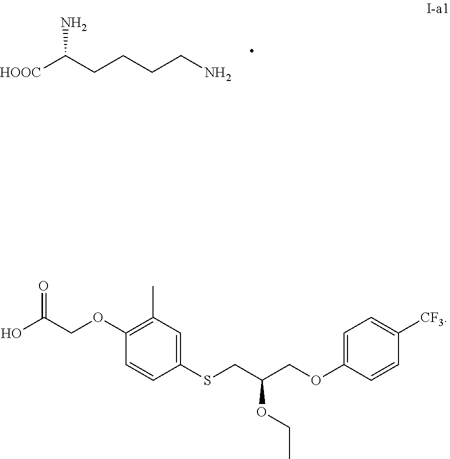 [SMILES: NCCCC[C@@H](N)C(=O)O ; CCO[C@H](COc1ccc(C(F)(F)F)cc1)CSc1ccc(OCC(=O)O)c(C)c1]

In some embodiments, the ratio of ethanol/methanol/water is about 80:about 20:about 1.

In some embodiments, the total amount of ethanol/methanol/water is about 15 to 25 volumes. In some embodiments, the total amount of ethanol/methanol/water is about 20 volumes.

In some embodiments, the recrystallization comprises a precipitation cycle at about 40 to 60° C., a heat cycle at about 50 to 70° C., a hold cycle, and a cooling cycle at about 0.1 to 1° C./minute.

In some embodiments, the recrystallization may be repeated as needed to yield the recrystallized lysine salt of a compound of formula I-a2.

In some embodiments, the recrystallized lysine salt of a compound of formula I-a2 is hydrated under conditions sufficient to form a hydrated lysine salt of a compound of formula I-b:

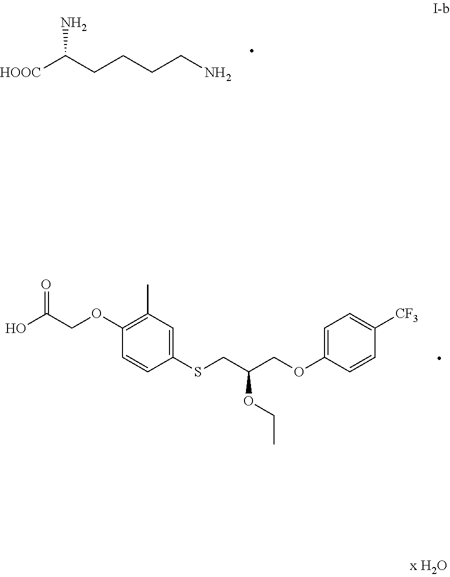

wherein x is variable. In some embodiments, the hydrated lysine salt of a compound of formula I-b is a channel hydrate. In a channel hydrate, water molecules may be either stoichiometric or non-stoichiometric with respect to the compound. In some embodiments, x is between about 1 and about 3. In some embodiments, x is about 2. In some embodiments, the water content of a composition comprising a compound described herein is about 3.5% to about 8.5% w/w.

In some embodiments, the recrystallized lysine salt of a compound of formula I-a2 is hydrated under conditions sufficient to form a hydrated lysine salt of a compound of formula I-b, wherein x is between about 1 and about 3.

In some embodiments, the hydrated lysine salt of a compound of formula I-b is a compound of formula I-b1:

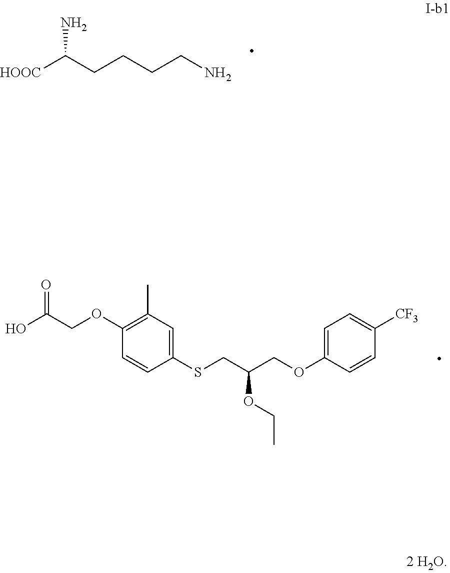 [SMILES: NCCCC[C@@H](N)C(=O)O ; CCO[C@H](COc1ccc(C(F)(F)F)cc1)CSc1ccc(OCC(=O)O)c(C)c1]

In some embodiments, the hydration occurs in an environmental chamber. In some embodiments, the temperature is about 20 to 45° C., and the relative humidity is about 30 to about 75%. In some embodiments, the temperature is about 25 to 40° C., and the relative humidity is about 35 to about 70%. In some embodiments, the temperature is about 25 to 35° C., and the relative humidity is about 60 to about 70%.

Some embodiments provide for a method of making a compound of formula I:

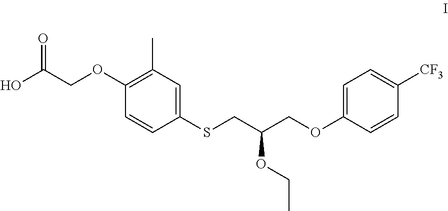 [SMILES: CCO[C@H](COc1ccc(C(F)(F)F)cc1)CSc1ccc(OCC(=O)O)c(C)c1]

or a pharmaceutically acceptable salt or hydrate thereof,
comprising:
(1) contacting a compound of formula (D):

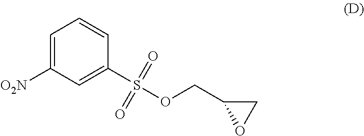 [SMILES: O=[N+]([O-])c1cccc(S(=O)(=O)OC[C@@H]2CO2)c1]

with a compound of formula (E):

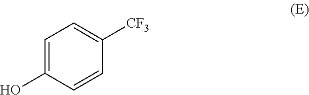 [SMILES: Oc1ccc(C(F)(F)F)cc1]

in the presence of a base under conditions sufficient to form a compound of formula (B):

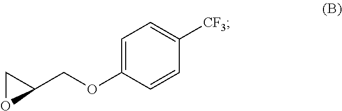 [SMILES: FC(F)(F)c1ccc(OC[C@@H]2CO2)cc1]

(2) contacting a compound of formula (F):

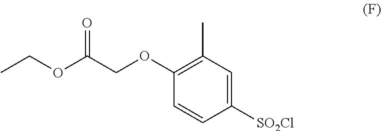 [SMILES: CCOC(=O)COc1ccc(S(=O)(=O)Cl)cc1C]

with a reducing agent under conditions sufficient to form a compound of formula (C):

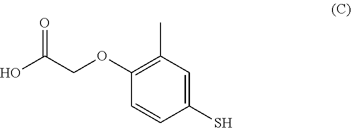

or a salt thereof;
(3) contacting a compound of formula (B) with a compound of formula (C), or a salt thereof, under conditions sufficient to form a compound of formula (A):

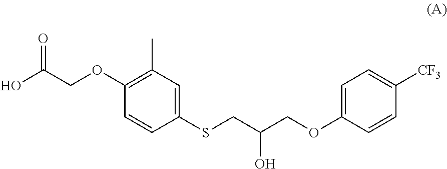 [SMILES: Cc1cc(SCC(O)COc2ccc(C(F)(F)F)cc2)ccc1OCC(=O)O]

or a salt thereof; and
(4) contacting a compound of formula (A), or a salt thereof, with CH₃CH₂—Br under conditions sufficient to form a compound of formula I or a pharmaceutically acceptable salt or hydrate thereof.

In some embodiments, the reaction conditions of steps (1)-(4) may be as described herein.

Some embodiments provide for a method of making a compound of formula I:

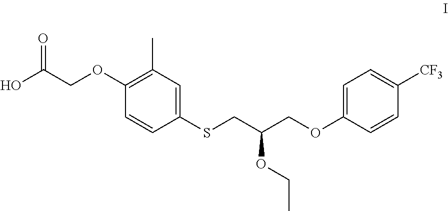 [SMILES: CCO[C@H](COc1ccc(C(F)(F)F)cc1)CSc1ccc(OCC(=O)O)c(C)c1]

or a pharmaceutically acceptable salt or hydrate thereof,
comprising:
(1) contacting a compound of formula (D):

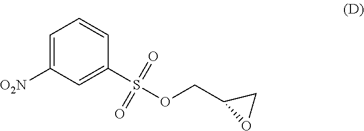 [SMILES: O=[N+]([O-])c1cccc(S(=O)(=O)OC[C@@H]2CO2)c1]

with a compound of formula (E):

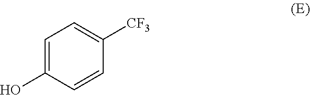 [SMILES: Oc1ccc(C(F)(F)F)cc1]

in the presence of a base under conditions sufficient to form a compound of formula (B):

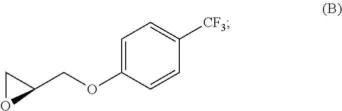 [SMILES: FC(F)(F)c1ccc(OC[C@@H]2CO2)cc1]

(2) contacting a compound of formula (F):

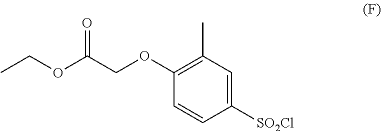 [SMILES: CCOC(=O)COc1ccc(S(=O)(=O)Cl)cc1C]

with a reducing agent under conditions sufficient to form a compound of formula (C):

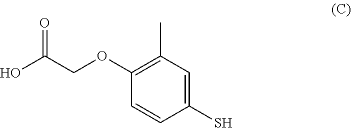

or a salt thereof;
(3) contacting a compound of formula (B) with a compound of formula (C), or a salt thereof, under conditions sufficient to form a compound of formula (A):

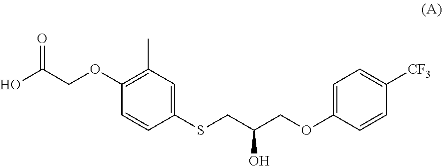 [SMILES: Cc1cc(SC[C@H](O)COc2ccc(C(F)(F)F)cc2)ccc1OCC(=O)O]

or a salt thereof; and
(4) contacting a compound of formula (A), or a salt thereof, with CH₃CH₂—Br in the presence of sodium hydroxide and dimethylsulfoxide (DMSO) and water under conditions sufficient to form a compound of formula I or a pharmaceutically acceptable salt or hydrate thereof.

Some embodiments provide for a method of making a hydrated lysine salt of a compound of formula I-b:

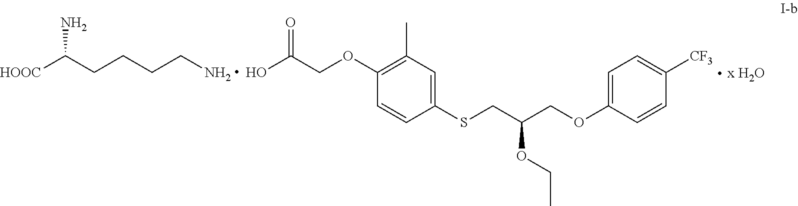

wherein x is between about 1 and about 3, comprising:
(1) contacting a compound of formula (D):

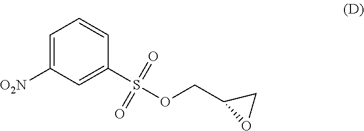 [SMILES: O=[N+]([O-])c1cccc(S(=O)(=O)OC[C@@H]2CO2)c1]

with a compound of formula (E):

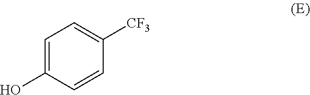 [SMILES: Oc1ccc(C(F)(F)F)cc1]

in the presence of a base under conditions sufficient to form a compound of formula (B):

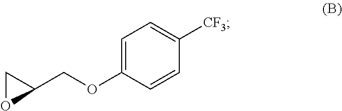 [SMILES: FC(F)(F)c1ccc(OC[C@@H]2CO2)cc1]

(2) contacting a compound of formula (F):

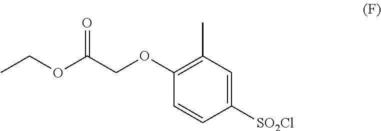 [SMILES: CCOC(=O)COc1ccc(S(=O)(=O)Cl)cc1C]

with a reducing agent under conditions sufficient to form a compound of formula (C):

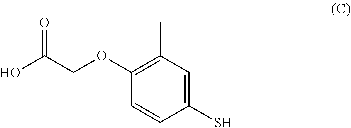

or a salt thereof;
(3) contacting a compound of formula (B) with a compound of formula (C), or a salt thereof, under conditions sufficient to form a compound of formula (A):

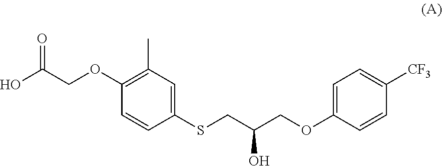 [SMILES: Cc1cc(SC[C@H](O)COc2ccc(C(F)(F)F)cc2)ccc1OCC(=O)O]

or a salt thereof;
(4) contacting a compound of formula (A), or a salt thereof, with CH₃CH₂—Br under conditions sufficient to form a compound of formula I;
(5) contacting a compound of formula I with L-lysine under conditions sufficient to form a lysine salt of a compound of formula I-a1:

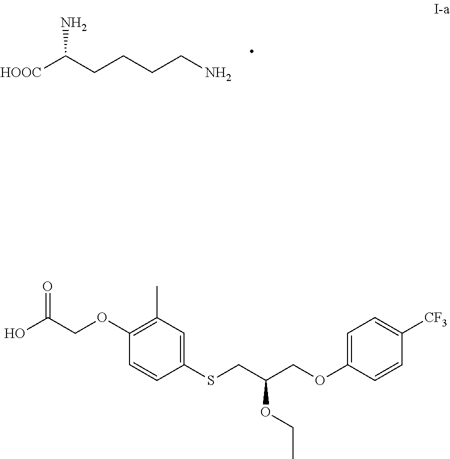 [SMILES: NCCCC[C@@H](N)C(=O)O ; CCO[C@H](COc1ccc(C(F)(F)F)cc1)CSc1ccc(OCC(=O)O)c(C)c1]

(6) contacting the lysine salt of a compound of formula I-a1 with methanol, ethanol, water, or a mixture thereof, under conditions sufficient to form a recrystallized lysine salt of a compound of formula I-a2:

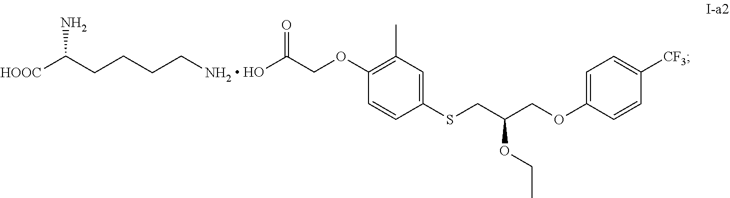 [SMILES: NCCCC[C@@H](N)C(=O)O ; CCO[C@H](COc1ccc(C(F)(F)F)cc1)CSc1ccc(OCC(=O)O)c(C)c1]

and (7) hydrating the recrystallized lysine salt of a compound of formula I-a2 under conditions sufficient to form a hydrated lysine salt of a compound of formula I-b.

In some embodiments, the reaction conditions of steps (1)-(7) may be as described herein.

### Compositions

Provided herein are compositions comprising seladelpar, or a salt thereof, or a pharmaceutically acceptable salt thereof, or hydrate thereof, or intermediates thereof, that may be prepared according to the processes and methods described herein.

It is contemplated that the processes described herein are able to achieve high purity of the compositions as described herein.

Provided herein is a composition comprising a compound of formula I:

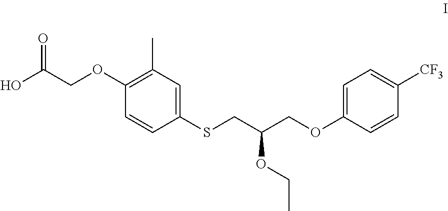 [SMILES: CCO[C@H](COc1ccc(C(F)(F)F)cc1)CSc1ccc(OCC(=O)O)c(C)c1]

or a pharmaceutically acceptable salt or hydrate thereof,
wherein the composition comprises less than about 2% a/a of a compound selected from:

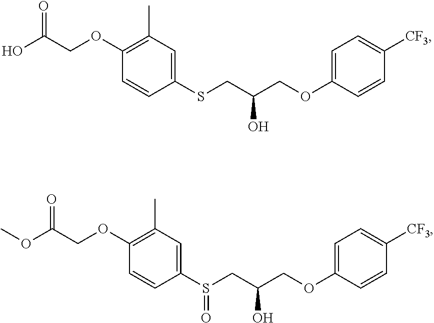 [SMILES: Cc1cc(SC[C@H](O)COc2ccc(C(F)(F)F)cc2)ccc1OCC(=O)O ; COC(=O)COc1ccc(S(=O)C[C@H](O)COc2ccc(C(F)(F)F)cc2)cc1C]

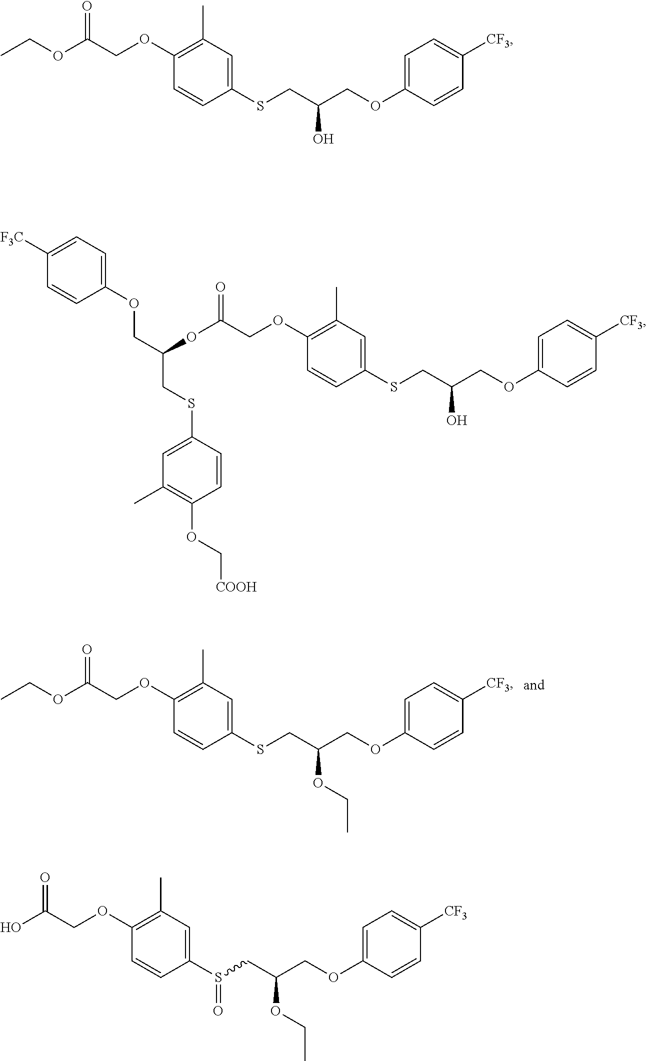 [SMILES: CCOC(=O)COc1ccc(SC[C@H](O)COc2ccc(C(F)(F)F)cc2)cc1C ; Cc1cc(SC[C@@H](COc2ccc(C(F)(F)F)cc2)OC(=O)COc2ccc(SC[C@H](O)COc3ccc(C(F)(F)F)cc3)cc2C)ccc1OCC(=O)O ; CCOC(=O)COc1ccc(SC[C@@H](COc2ccc(C(F)(F)F)cc2)OCC)cc1C ; CCO[C@H](COc1ccc(C(F)(F)F)cc1)CS(=O)c1ccc(OCC(=O)O)c(C)c1]

or a combination thereof.

Provided herein is a composition comprising a lysine salt of a compound of formula I-a:

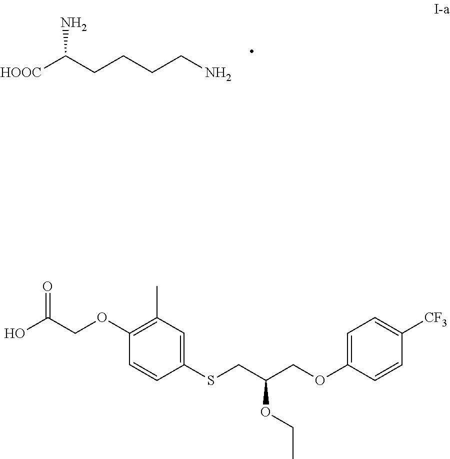 [SMILES: NCCCC[C@@H](N)C(=O)O ; CCO[C@H](COc1ccc(C(F)(F)F)cc1)CSc1ccc(OCC(=O)O)c(C)c1]

wherein the composition comprises less than about 2% a/a of a compound selected from:

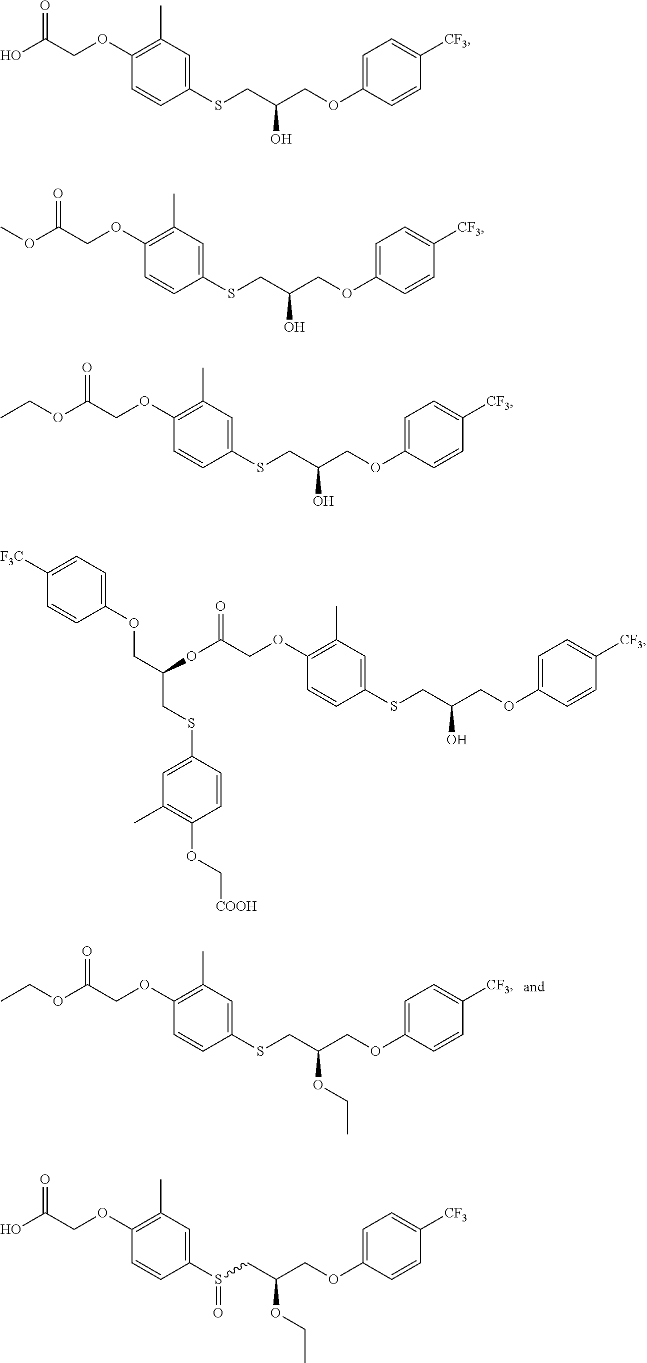 [SMILES: Cc1cc(SC[C@H](O)COc2ccc(C(F)(F)F)cc2)ccc1OCC(=O)O ; COC(=O)COc1ccc(SC[C@H](O)COc2ccc(C(F)(F)F)cc2)cc1C ; CCOC(=O)COc1ccc(SC[C@H](O)COc2ccc(C(F)(F)F)cc2)cc1C ; Cc1cc(SC[C@@H](COc2ccc(C(F)(F)F)cc2)OC(=O)COc2ccc(SC[C@H](O)COc3ccc(C(F)(F)F)cc3)cc2C)ccc1OCC(=O)O ; CCOC(=O)COc1ccc(SC[C@@H](COc2ccc(C(F)(F)F)cc2)OCC)cc1C ; CCO[C@H](COc1ccc(C(F)(F)F)cc1)CS(=O)c1ccc(OCC(=O)O)c(C)c1]

or a combination thereof.

In some embodiments, the composition comprises less than about 2% a/a, or less than about 1% a/a of a compound selected from:

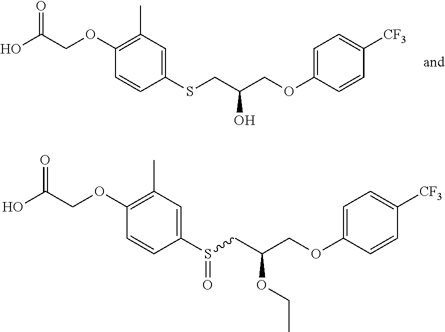 [SMILES: Cc1cc(SC[C@H](O)COc2ccc(C(F)(F)F)cc2)ccc1OCC(=O)O ; CCO[C@H](COc1ccc(C(F)(F)F)cc1)CS(=O)c1ccc(OCC(=O)O)c(C)c1]

or a combination thereof.

Some embodiments provide for a composition comprising a compound of formula I, or a pharmaceutically acceptable salt or hydrate thereof, wherein the composition comprises at least about 97% w/w (weight by weight) of the compound of formula I, or a pharmaceutically acceptable salt or hydrate thereof. In some embodiments, the composition comprises at least about 98% w/w of the compound of formula I, or a pharmaceutically acceptable salt or hydrate thereof.

Some embodiments provide for composition comprising a compound of formula I-b1:

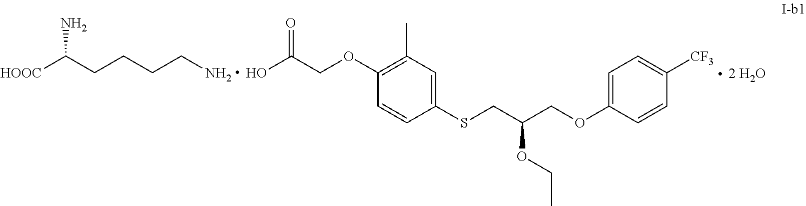 [SMILES: NCCCC[C@@H](N)C(=O)O ; CCO[C@H](COc1ccc(C(F)(F)F)cc1)CSc1ccc(OCC(=O)O)c(C)c1]

wherein the composition comprises at least about 97% w/w of the compound of formula I-b1. In some embodiments, the composition comprises at least about 98% w/w of the compound of formula I-b1.

In some embodiments, the composition comprises less than about 2% w/w, less than about 1.5% w/w, or less than about 1% w/w of a compound selected from:

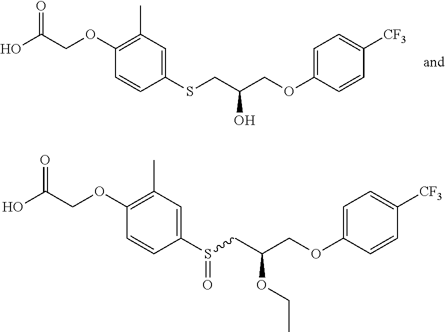 [SMILES: Cc1cc(SC[C@H](O)COc2ccc(C(F)(F)F)cc2)ccc1OCC(=O)O ; CCO[C@H](COc1ccc(C(F)(F)F)cc1)CS(=O)c1ccc(OCC(=O)O)c(C)c1]

or a combination thereof.

Some embodiments provide for a composition comprising a compound of formula I-b1:

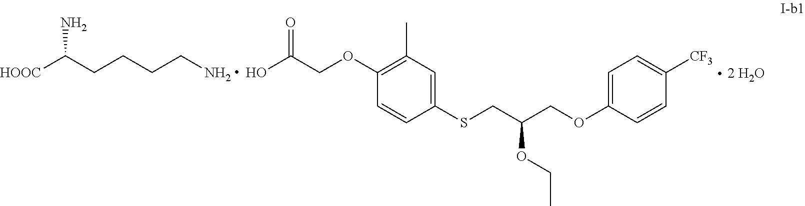 [SMILES: NCCCC[C@@H](N)C(=O)O ; CCO[C@H](COc1ccc(C(F)(F)F)cc1)CSc1ccc(OCC(=O)O)c(C)c1]

wherein the composition comprises at least about 97% w/w of the compound of formula I-b1, and less than about 2% w/w of a compound selected from:

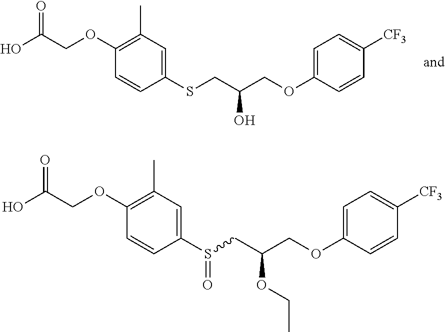 [SMILES: Cc1cc(SC[C@H](O)COc2ccc(C(F)(F)F)cc2)ccc1OCC(=O)O ; CCO[C@H](COc1ccc(C(F)(F)F)cc1)CS(=O)c1ccc(OCC(=O)O)c(C)c1]

or a combination thereof.

Some embodiments provide for a composition comprising a compound of formula I-b1, wherein the composition comprises at least about 98% w/w of the compound of formula I-b1, and less than about 2% w/w of a compound selected from:

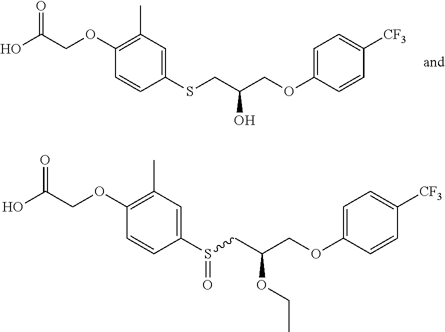 [SMILES: Cc1cc(SC[C@H](O)COc2ccc(C(F)(F)F)cc2)ccc1OCC(=O)O ; CCO[C@H](COc1ccc(C(F)(F)F)cc1)CS(=O)c1ccc(OCC(=O)O)c(C)c1]

or a combination thereof.

Some embodiments provide for a composition comprising a compound of formula I-b1, wherein the composition comprises at least about 98% w/w of the compound of formula I-b1, and less than about 1.5% w/w of a compound selected from:

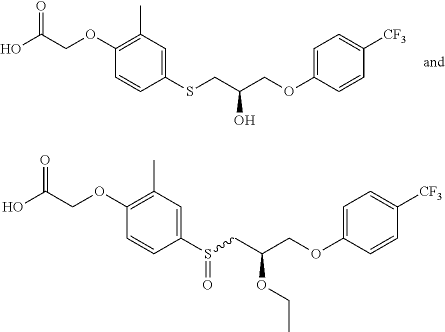 [SMILES: Cc1cc(SC[C@H](O)COc2ccc(C(F)(F)F)cc2)ccc1OCC(=O)O ; CCO[C@H](COc1ccc(C(F)(F)F)cc1)CS(=O)c1ccc(OCC(=O)O)c(C)c1]

or a combination thereof.

In some embodiments, the composition comprises at least about 98% w/w of the compound of formula I-b1, and less than about 1% w/w of a compound selected from:

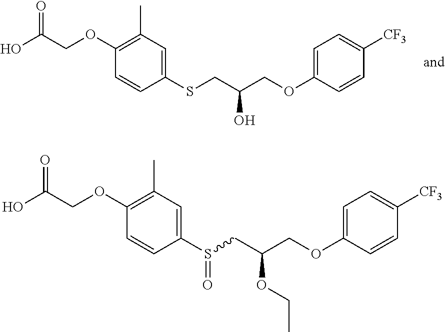 [SMILES: Cc1cc(SC[C@H](O)COc2ccc(C(F)(F)F)cc2)ccc1OCC(=O)O ; CCO[C@H](COc1ccc(C(F)(F)F)cc1)CS(=O)c1ccc(OCC(=O)O)c(C)c1]

or a combination thereof.

Also provided herein is a composition comprising a compound of formula I:

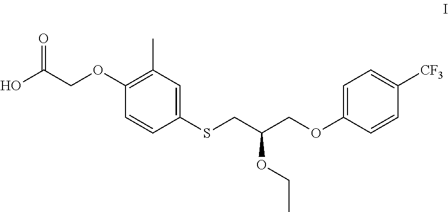 [SMILES: CCO[C@H](COc1ccc(C(F)(F)F)cc1)CSc1ccc(OCC(=O)O)c(C)c1]

or a pharmaceutically acceptable salt or a hydrate thereof,
wherein the composition comprises less than 0.1% a/a of a compound of formula II:

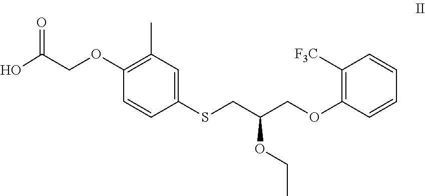 [SMILES: CCO[C@H](COc1ccccc1C(F)(F)F)CSc1ccc(OCC(=O)O)c(C)c1]

or a pharmaceutically acceptable salt thereof.

Provided herein is a composition comprising a compound of formula I-b1:

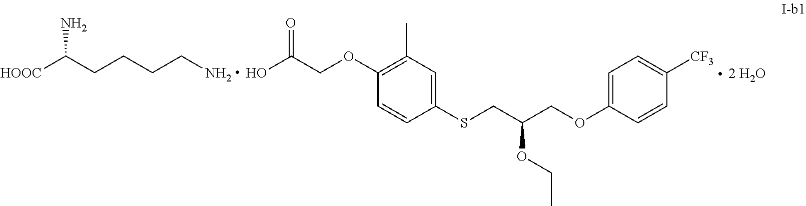 [SMILES: NCCCC[C@@H](N)C(=O)O ; CCO[C@H](COc1ccc(C(F)(F)F)cc1)CSc1ccc(OCC(=O)O)c(C)c1]

wherein the composition comprises less than 0.1% a/a of a compound of formula II-a:

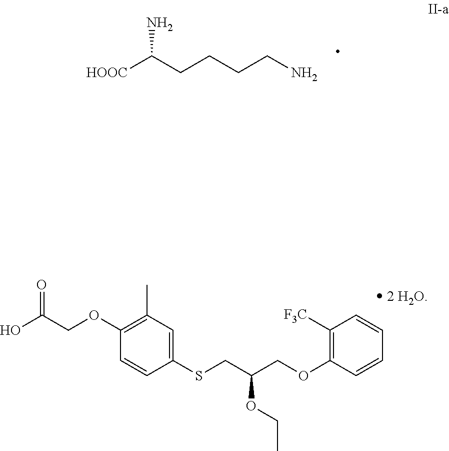 [SMILES: NCCCC[C@@H](N)C(=O)O ; CCO[C@H](COc1ccccc1C(F)(F)F)CSc1ccc(OCC(=O)O)c(C)c1]

In some embodiments, the composition comprises less than about 0.09% a/a of a compound of formula II-a. In some embodiments, the composition comprises less than about 0.08% a/a of a compound of formula II-a. In some embodiments, the composition comprises less than about 0.07% a/a of a compound of formula II-a. In some embodiments, the composition comprises less than about 0.06% a/a of a compound of formula II-a. In some embodiments, the composition comprises less than about 0.05% a/a of a compound of formula II-a.

In some embodiments, the amount of components described herein in the compositions described herein may be determined according to methods known in the art, such as gas chromatography (GC), high pressure liquid chromatography (HPLC), ultra-high performance liquid chromatography (UHPLC), and the like.

In some embodiments, the compositions described herein further comprise a pharmaceutically acceptable carrier or pharmaceutically acceptable excipient.

Some embodiments provide for methods of treating primary biliary cholangitis (PBC) in a subject in need thereof comprising administering a therapeutically effective amount of a composition as described herein, optionally wherein the subject is unable to tolerate ursodeoxycholic acid (UDCA).

Some embodiments provide for methods of treating primary biliary cholangitis (PBC) in a subject in need thereof comprising administering a therapeutically effective amount of a composition as described herein in combination with ursodeoxycholic acid (UDCA), wherein the subject has an inadequate response to UDCA.

It is appreciated that certain features described herein, which are, for clarity, described in the context of separate embodiments, may also be provided in combination in a single embodiment. Conversely, various features described herein, which are, for brevity, described in the context of a single embodiment, may also be provided separately or in any suitable subcombination.

### EXAMPLES

The compounds of the disclosure may be prepared using methods disclosed herein and routine modifications thereof which will be apparent given the disclosure herein and methods well known in the art. Conventional and well-known synthetic methods may be used in addition to the teachings herein. The synthesis of compounds described herein may be accomplished as described in the following examples. If available, reagents may be purchased commercially, e.g., from Sigma Aldrich or other chemical suppliers. Unless otherwise noted, the starting materials for the following reactions may be obtained from commercial sources.

### Example 1: Synthesis of Compound of Formula (B)

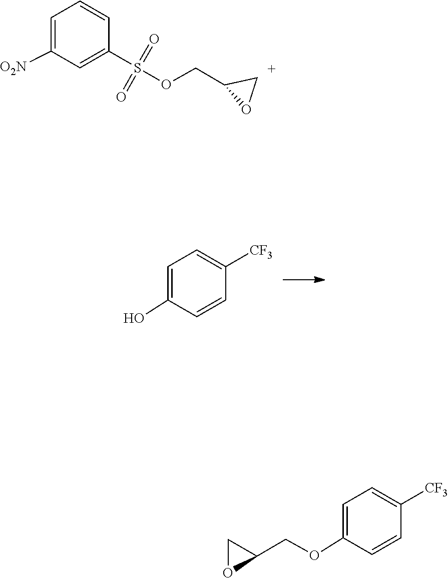 [SMILES: O=[N+]([O-])c1cccc(S(=O)(=O)OC[C@@H]2CO2)c1 ; Oc1ccc(C(F)(F)F)cc1 ; FC(F)(F)c1ccc(OC[C@@H]2CO2)cc1]

(S)-(+)-Glycidyl Nosylate (compound of formula (D)), which serves as the scale factor for the process, and 4-(Trifluoromethyl) phenol (compound of formula (E)) were mixed in dimethylsulfoxide at 15-25° C. until a solution was observed. Cesium carbonate was added slowly, and the mixture was then held at 15° C. to 25° C. until the reaction was complete (at least 2 hours). Water and heptane were added, and the mixture was stirred for more than about 10 minutes. The organic layer was washed twice with water and then clarified through a 1.2 μm filter. The heptane solution was then concentrated in vacuo at an internal temperature of less than or equal to about 35° C. to the target volume (about 0.63 L/kg of compound of formula (D)). Additional heptane was added, and the resulting solution was again concentrated in vacuo to the target volume. This process was then repeated once, and the product was obtained as an oil. IR of the product conformed to reference spectrum; and the product was confirmed by ¹H NMR. ≥98% purity as measured by UHPLC was achieved.

### Example 2: Synthesis of Compound of Formula (C)

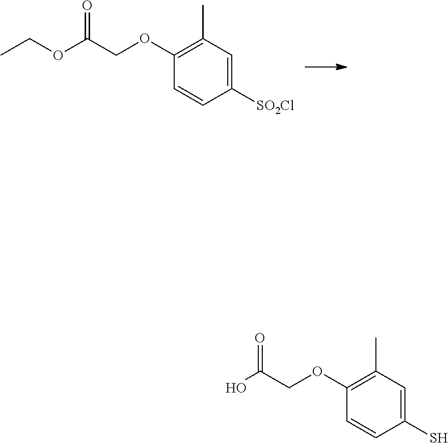 [SMILES: CCOC(=O)COc1ccc(S(=O)(=O)Cl)cc1C ; Cc1cc(S)ccc1OCC(=O)O]

Ethyl 2-(4-chlorosulfonyl-2-methylphenoxy)acetate (compound of formula (F)), which serves as the scale factor for the process, and tin powder were added to a reactor. Acetic acid was added, and stirring was initiated. Water and hydrochloric acid were then charged, and the reaction was heated to 55° C. to 65° C. and held at 55° C. to 65° C. until the reaction was complete (at least 4 hours). The reaction mixture was then filtered into a clean reactor to remove excess tin and tin salts. A solution of 3 N HCl was then added to the hot reaction mixture filtrate, and the temperature was adjusted to 15° C. to 25° C. and held for at least 16 hours. The slurry was then transferred to a filter dryer to collect the solids, washed in portions with 3 N HCl, then with water twice, and dried under vacuum at 35° C. to 45° C. until a loss on drying of ≤1%, yielding the product as a solid. FTIR of the product conformed to reference spectrum; and the product was confirmed by 1H NMR. ≥98% purity as measured by UHPLC was achieved.

### Example 3: Synthesis of Compound of Formula (A)

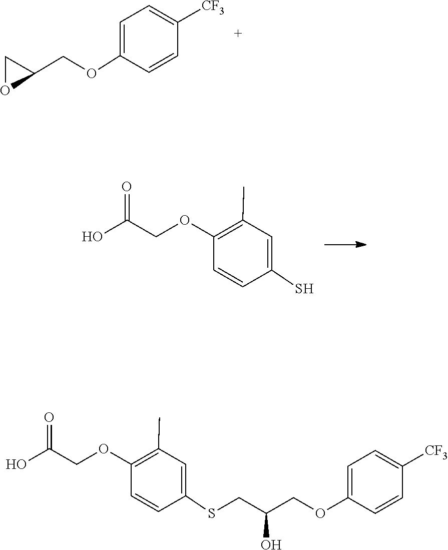 [SMILES: FC(F)(F)c1ccc(OC[C@@H]2CO2)cc1 ; Cc1cc(S)ccc1OCC(=O)O ; Cc1cc(SC[C@H](O)COc2ccc(C(F)(F)F)cc2)ccc1OCC(=O)O]

2-(4-mercapto-2-methylphenoxy) acetic acid (compound of formula (C)), which serves as the scale factor for the process, and DMF were charged to the reactor. (S)-2-((4-(trifluoromethyl) phenoxy)methyl) oxirane (compound of formula (B)) was added under agitation. Additional DMF was then added as a rinse. The reactor was purged three times by pulling vacuum to ≤5 psia, and then the vacuum was slowly broken with nitrogen at about 16 psia. The temperature was adjusted to 15 to 25° C., and triethylamine was slowly charged while controlling the temperature at ≤50° C. The temperature was adjusted to 25 to 35° C., and the reaction mixture was stirred at this temperature range for at least 4 hours. The reaction was quenched with water and treated with 50% NaOH, and the basic aqueous layer was extracted with methyl tert-butyl ether (MTBE). The basic aqueous layer was separated, washed with MTBE, and acidified with concentrated HCl, and the crude product was extracted into MTBE and washed with water twice. The organic phase was concentrated to an oil and then twice dissolved in methanol (MeOH) and concentrated to achieve azeotropic removal of MTBE. The oil was dissolved in MeOH, and water was added at 30° C. Crystallization was carried out at 20° C. The purified solids were filtered, rinsed with MeOH/water, and dried in vacuo to afford the product as a solid. FTIR of the product conformed to reference spectrum; and the product was confirmed by ¹H NMR and LCMS. ≥98% purity as measured by UHPLC was achieved.

### Example 4: Synthesis of Compound of Formula I

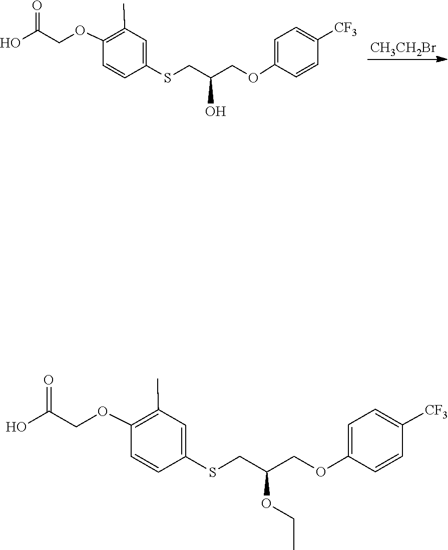

Compound of formula (A), which serves as the scale factor for the process, and DMSO were charged to a reactor. Agitation was started at 90 to 115 rpm, and ethyl bromide was charged, followed by a rinse with DMSO. The internal temperature was adjusted to 15 to 25° C. (target 15° C.), and the mixture was held until a solution was obtained. NaOH (50% solution) was charged as fast as practical while maintaining an internal temperature between 15 to 40° C., followed by a rinse with water. The temperature was adjusted to 15 to 25° C. (target 20° C.), and the mixture was held stirring at this temperature for 1 to 4 hours or until reaction was deemed complete.

The reaction mixture was diluted with water, and the reactor contents were stirred at 90 to 115 rpm for up to 1 hour. HCl was charged to the reaction mixture while monitoring the pH until a pH of less than about 3 was obtained. MTBE was charged to the reaction vessel, and the two-phase mixture was stirred to up to about 1 hour at 90 to 115 rpm. Then, stirring was stopped, and the layers were allowed to separate for at least 15 minutes until a clear phase separation was observed. The organic layer was washed twice with water, stirring the bi-phasic mixture each time for up to 60 min at 90 to 115 rpm and then allowing the layers to separate for at least 15 minutes until a clear phase separation was observed. Stirring was resumed after removal of the aqueous layer was complete.

The resulting organic phase was concentrated to a target volume of 4 L/kg of a compound of formula (A) by distilling the reactor contents under vacuum while maintaining the jacket temperature≤50° C., and the internal temperature≤40° C., and then the vacuum was released using nitrogen. Ethanol reconstitution and batch concentration were performed twice by charging ethanol and concentrating the mixture with the same initial parameters to the target volume of 4 L/kg of a compound of formula (A) each time.

The product was used in the next step without further purification.

### Example 5: Synthesis of a Lysine Salt of a Compound of Formula I-a1

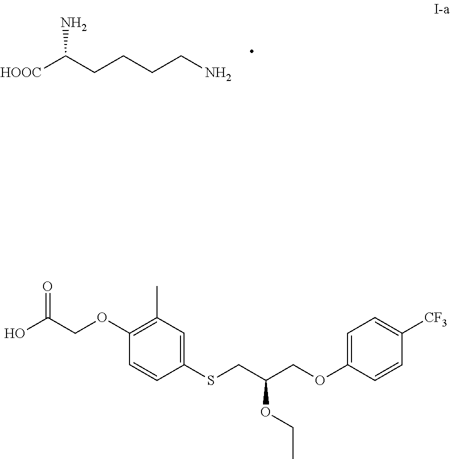 [SMILES: NCCCC[C@@H](N)C(=O)O ; CCO[C@H](COc1ccc(C(F)(F)F)cc1)CSc1ccc(OCC(=O)O)c(C)c1]

Ethanol and methanol were charged to the reactor. L-lysine hydrate and water were charged to the reactor.

The internal temperature of the reactor was adjusted to 70 to 80° C. The mixture was stirred at 90 to 115 rpm for 15 to 60 minutes, then adjusted to 30 to 40° C. (target 35° C.), and held at this temperature and stirred until precipitation was observed. The contents of the reactor were then heated to 55 to 65° C. (target 60° C.), stirred at this temperature for 2 to 3 hours, and then cooled to 15 to 25° C. (target 20° C.) over 375 to 425 min (target about 0.1° C./min). The mixture was then stirred at 15 to 25° C. (target 20° C.) for 1 to 24 hours.

The slurry was transferred to a filter dryer, and the solids were deliquored using nitrogen pressure. The reactor was then rinsed with an ethanol-methanol rinse solution, prepared by mixing ethanol and methanol. The rinse was then transferred to the filter dryer and used to wash the solids. The wash was displaced using nitrogen pressure. A second ethanol-methanol rinse solution was prepared and again transferred to the filter dryer, displacing the wash using nitrogen pressure. The solids were then dried by lowering the agitator to the lowest position and stirring under with a slight nitrogen purge and a jacket supply temperature of 45 to 55° C. for 12 to 48 hours. Drying was continued until a loss on drying (LOD) of ≤5%. The solids were stirred with the agitator prior to removing the sample to ensure a uniform sample was obtained. The product was used in the next step and confirmed by HPLC.

### Example 6: Preparation of a Recrystallized Lysine Salt of a Compound of Formula I-a2

A compound of formula I-a1, which serves as the scale factor for the process, was dissolved in ethanol:methanol:water at reflux. The mixture was then polish filtered through a clarification filter and heated until a solution was observed. Seladelpar seeds (which can be made according to methods described herein or known in the art) were charged to the polish filtered solution at 60 to 65° C. to initiate crystallization. The mixture was inspected for the presence of seeds, and once the presence of the seeds was confirmed, the mixture was held under stirring at 60 to 65° C. for about 2-3 hours. The seeded mixture was then slowly cooled to about 15 to 25° C. over 7-9 hours (target about 0.1° C./minute). Agitation was adjusted to the lowest possible rate as necessary to suspend the solids, and the reactor contents were stirred at this temperature range for 1-24 hours. The slurry was filtered, washed with ethanol:methanol, and then dried to yield a recrystallized lysine salt of a compound of formula I-a2. Retention time by HPLC was consistent with reference standard.

### Example 7: Preparation of a Hydrated Lysine Salt of a Compound of Formula I-b1

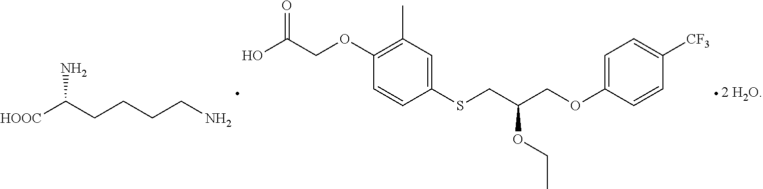 [SMILES: NCCCC[C@@H](N)C(=O)O ; CCO[C@H](COc1ccc(C(F)(F)F)cc1)CSc1ccc(OCC(=O)O)c(C)c1]

A recrystallized lysine salt of a compound of formula I-a2, which serves as the scale factor for the process and as prepared according to Example 6, was charged to hydration trays, calculating an approximate amount to be loaded in each tray (about 0.17 kg/kg of a compound of formula I-a2). The environmental chamber was then loaded with the full trays, and the tray positions recorded (shelves 1 to 6). The environmental chamber set point was adjusted to a temperature of 30° C. and a humidity of 65% relative humidity (RH), and the product was hydrated for at least 36 hours while maintaining a temperature of about 25 to 35° C. and humidity of 45 to 75% RH (set point of 30° C. and humidity of 65% RH). After completing the first hydration cycle, using a spatula to turn over the product in all 6 trays, the trays were re-arranged by replacing the tray from shelf 6 on shelf 1 and the tray from shelf 1 on shelf 6.

A second hydration cycle was performed with the product in the new shelf locations while maintaining the same conditions, temperature of 25 to 35° C. and humidity of 45 to 75% RH (set point of 30° C. and humidity of 65% RH), and the hydration was continued for at least 36 hours, yielding the product. IR of the product conformed to reference spectrum; ≥98% purity as measured by UHPLC was achieved.

Unless otherwise defined, all technical and scientific terms used herein have the same meaning as commonly understood by one of ordinary skill in the art to which this disclosure belongs.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the embodiments claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A method of making a compound of formula I: 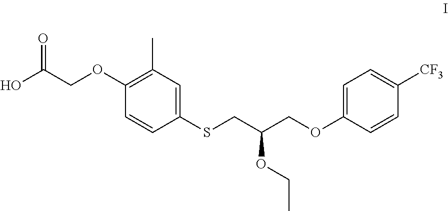 [SMILES: CCO[C@H](COc1ccc(C(F)(F)F)cc1)CSc1ccc(OCC(=O)O)c(C)c1]
or a pharmaceutically acceptable salt or hydrate thereof,
comprising:
contacting a compound of formula (A): 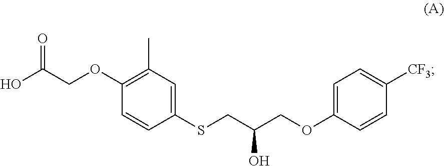 [SMILES: Cc1cc(SC[C@H](O)COc2ccc(C(F)(F)F)cc2)ccc1OCC(=O)O]
or a salt thereof,
with CH₃CH₂-LG, wherein LG is a leaving group,
under conditions sufficient to form a compound of formula I or a pharmaceutically acceptable salt or hydrate thereof.

2. The method of claim 1, comprising contacting a compound of formula (A), or a salt thereof, with CH₃CH₂-LG in the presence of sodium hydroxide and dimethylsulfoxide (DMSO) and water.

3. A method of making a compound of formula I: 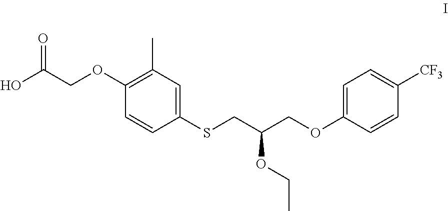 [SMILES: CCO[C@H](COc1ccc(C(F)(F)F)cc1)CSc1ccc(OCC(=O)O)c(C)c1]
or a pharmaceutically acceptable salt or hydrate thereof,
comprising:
(a) contacting a compound of formula (B): 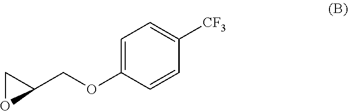 [SMILES: FC(F)(F)c1ccc(OC[C@@H]2CO2)cc1]
with a compound of formula (C): 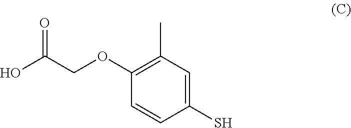
or a salt thereof,
under conditions sufficient to form a compound of formula (A): 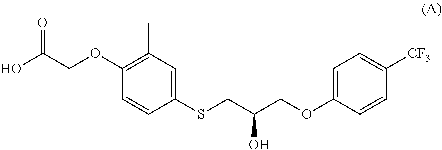 [SMILES: Cc1cc(SC[C@H](O)COc2ccc(C(F)(F)F)cc2)ccc1OCC(=O)O]
or a salt thereof;
and (b) contacting a compound of formula (A), or a salt thereof, with CH₃CH₂-LG, wherein LG is a leaving group,
under conditions sufficient to form a compound of formula I or a pharmaceutically acceptable salt or hydrate thereof.

4. The method of any one of claims 1-3, wherein LG is a halo, triflate, tosylate, or mesylate.

5. The method of any one of claims 1-4, wherein LG is Br.

6. The method of any one of claims 3-5, wherein step (a) comprises a base.

7. The method of claim 6, wherein the base is triethylamine.

8. The method of any one of claims 3-7, wherein step (b) comprises a base and a solvent.

9. The method of claim 8, wherein the base is sodium hydroxide, and the solvent is dimethylsulfoxide and water.

10. The method of any one of claims 3-9, comprising:
contacting a compound of formula (D): 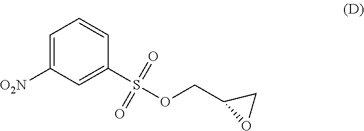 [SMILES: O=[N+]([O-])c1cccc(S(=O)(=O)OC[C@@H]2CO2)c1]
with a compound of formula (E): 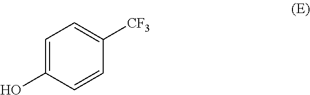 [SMILES: Oc1ccc(C(F)(F)F)cc1]
in the presence of a base under conditions sufficient to form a compound of formula (B).

11. The method of claim 10, wherein the base is cesium carbonate or potassium carbonate.

12. The method of claim 10, wherein the base is cesium carbonate.

13. The method of any one of claims 3-12, comprising:
contacting a compound of formula (F): 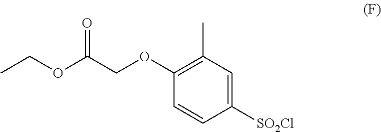 [SMILES: CCOC(=O)COc1ccc(S(=O)(=O)Cl)cc1C]
with a reducing agent under conditions sufficient to form a compound of formula (C) or a salt thereof.

14. The method of claim 13, wherein the reducing agent is tin powder in the presence of an acid.

15. The method of claim 14, wherein the acid is acetic acid, hydrochloric acid, or a mixture thereof.

16. A method of making a compound of formula I: 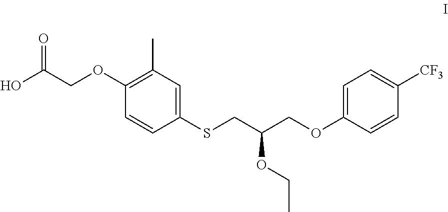 [SMILES: CCO[C@H](COc1ccc(C(F)(F)F)cc1)CSc1ccc(OCC(=O)O)c(C)c1]
or a pharmaceutically acceptable salt or hydrate thereof,
comprising:
(a) contacting a compound of formula (G): 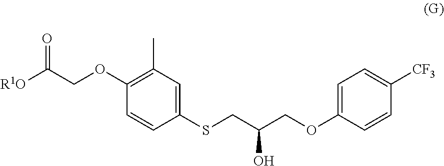
wherein R¹ is C₁₋₆ alkyl, C₂₋₆ alkenyl, C₂₋₆ alkynyl, C₃₋₁₀ cycloalkyl, heterocyclyl, aryl, or heteroaryl;
with CH₃CH₂-LG, wherein LG is a leaving group,
in the presence of sodium hydroxide and dimethylsulfoxide (DMSO) and water,
under conditions sufficient to form a compound of formula III: 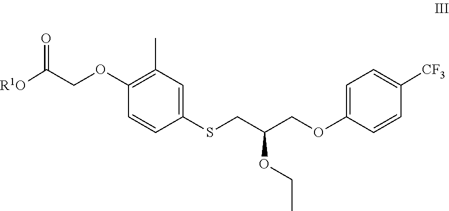
and (b) hydrolyzing the compound of formula III under conditions sufficient to form a compound of formula I or a pharmaceutically acceptable salt or hydrate thereof.

17. The method of claim 16, wherein LG is Br.

18. The method of any one of claims 16-17, wherein R¹ is C₁₄ alkyl.

19. The method of any one of the preceding claims, further comprising:
contacting a compound of formula I with L-lysine under conditions sufficient to form a lysine salt of a compound of formula I-a1:

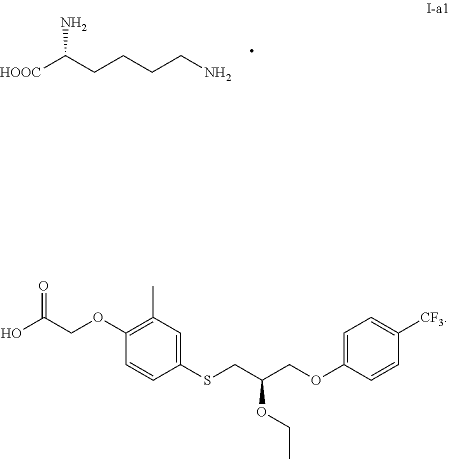 [SMILES: NCCCC[C@@H](N)C(=O)O ; CCO[C@H](COc1ccc(C(F)(F)F)cc1)CSc1ccc(OCC(=O)O)c(C)c1]

20. The method of claim 19, further comprising contacting the lysine salt of a compound of formula I-a1 with methanol, ethanol, water, or a mixture thereof, under conditions sufficient to form a recrystallized lysine salt of a compound of formula I-a2:

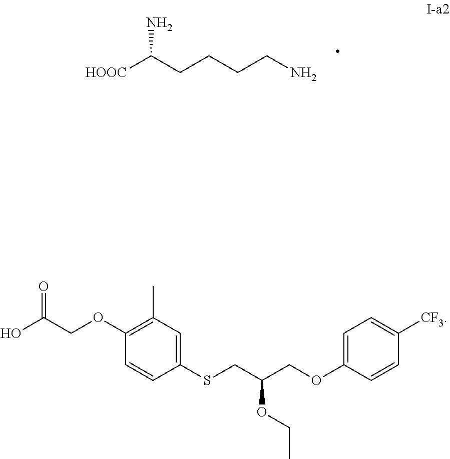 [SMILES: NCCCC[C@@H](N)C(=O)O ; CCO[C@H](COc1ccc(C(F)(F)F)cc1)CSc1ccc(OCC(=O)O)c(C)c1]

21. The method of claim 20, wherein the recrystallized lysine salt of a compound of formula I-a2 is hydrated under conditions sufficient to form a hydrated lysine salt of a compound of formula I-b: 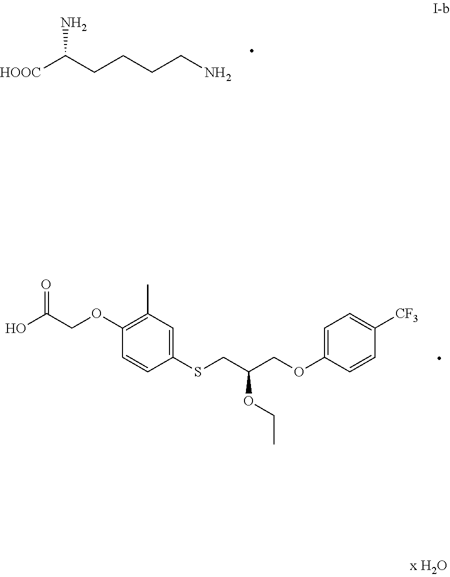
wherein x is between about 1 and about 3.

22. The method of claim 21, wherein the hydrated lysine salt of a compound of formula I-b is a compound of formula I-b1:

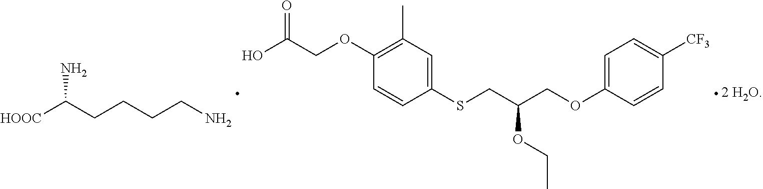 [SMILES: NCCCC[C@@H](N)C(=O)O ; CCO[C@H](COc1ccc(C(F)(F)F)cc1)CSc1ccc(OCC(=O)O)c(C)c1]

23. A composition comprising a lysine salt of a compound of formula I-a: 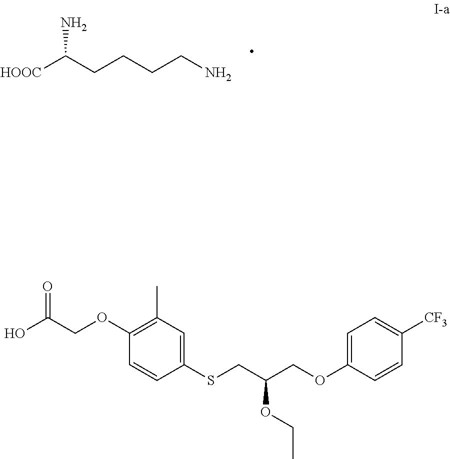 [SMILES: NCCCC[C@@H](N)C(=O)O ; CCO[C@H](COc1ccc(C(F)(F)F)cc1)CSc1ccc(OCC(=O)O)c(C)c1]
wherein the composition comprises less than about 2% a/a of a compound selected from: 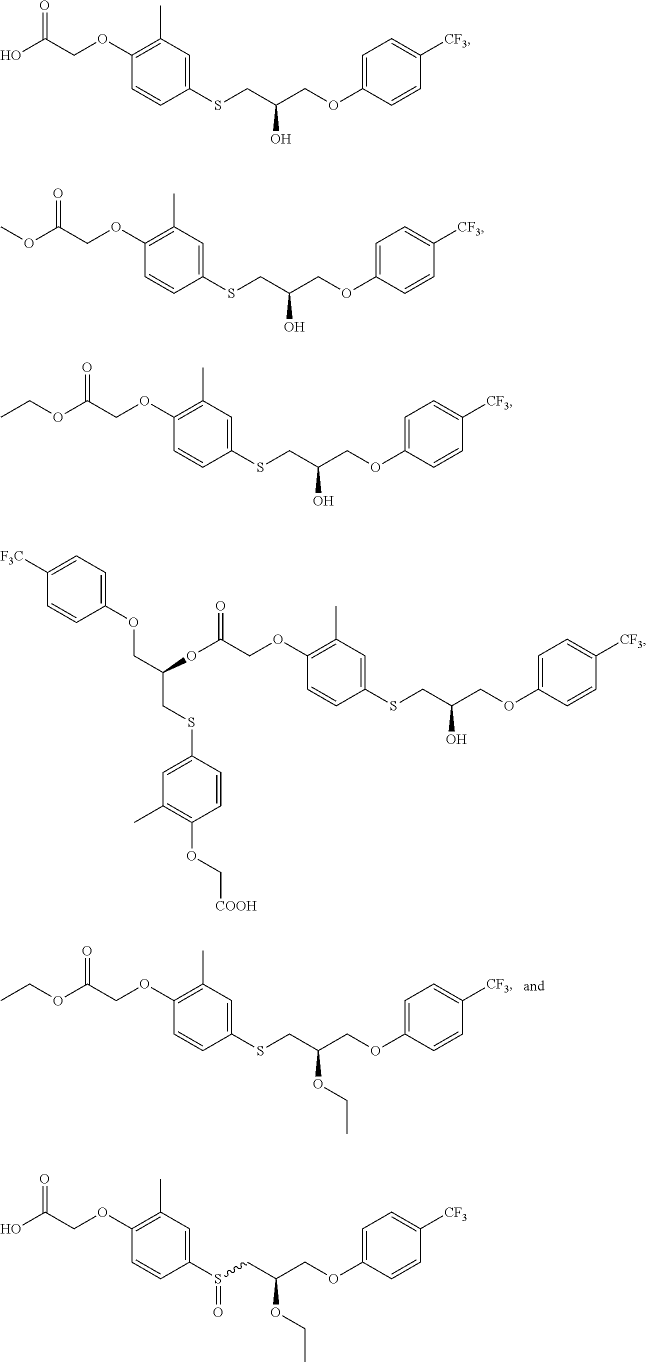 [SMILES: Cc1cc(SC[C@H](O)COc2ccc(C(F)(F)F)cc2)ccc1OCC(=O)O ; COC(=O)COc1ccc(SC[C@H](O)COc2ccc(C(F)(F)F)cc2)cc1C ; CCOC(=O)COc1ccc(SC[C@H](O)COc2ccc(C(F)(F)F)cc2)cc1C ; Cc1cc(SC[C@@H](COc2ccc(C(F)(F)F)cc2)OC(=O)COc2ccc(SC[C@H](O)COc3ccc(C(F)(F)F)cc3)cc2C)ccc1OCC(=O)O ; CCOC(=O)COc1ccc(SC[C@@H](COc2ccc(C(F)(F)F)cc2)OCC)cc1C ; CCO[C@H](COc1ccc(C(F)(F)F)cc1)CS(=O)c1ccc(OCC(=O)O)c(C)c1]
or a combination thereof.

24. A composition comprising a compound of formula I: 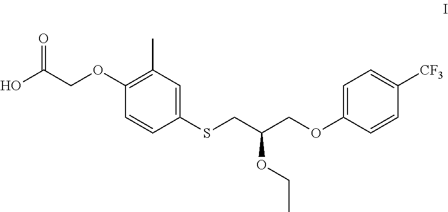 [SMILES: CCO[C@H](COc1ccc(C(F)(F)F)cc1)CSc1ccc(OCC(=O)O)c(C)c1]
or a pharmaceutically acceptable salt or hydrate thereof,
wherein the composition comprises less than 0.1% a/a of a compound of formula II: 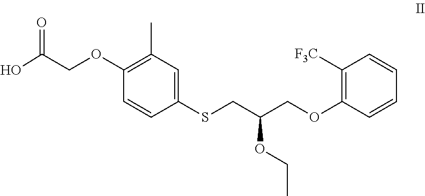 [SMILES: CCO[C@H](COc1ccccc1C(F)(F)F)CSc1ccc(OCC(=O)O)c(C)c1]
or a pharmaceutically acceptable salt thereof.

25. A composition comprising a compound of formula I-b1: 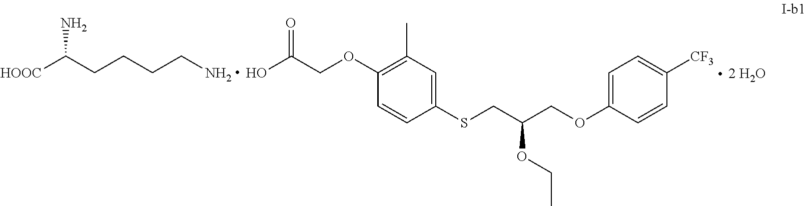 [SMILES: NCCCC[C@@H](N)C(=O)O ; CCO[C@H](COc1ccc(C(F)(F)F)cc1)CSc1ccc(OCC(=O)O)c(C)c1]
wherein the composition comprises less than 0.1% a/a of a compound of formula II-a:

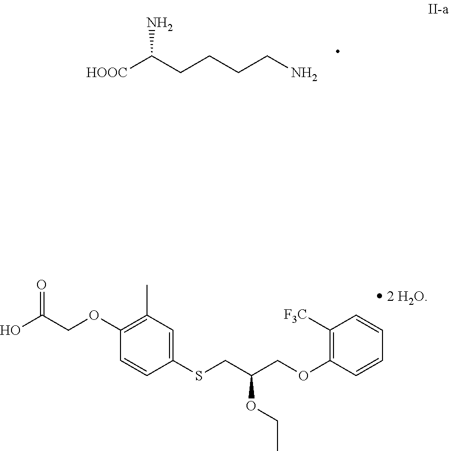 [SMILES: NCCCC[C@@H](N)C(=O)O ; CCO[C@H](COc1ccccc1C(F)(F)F)CSc1ccc(OCC(=O)O)c(C)c1]

26. The composition of claim 25, wherein the composition comprises less than about 0.07% a/a of a compound of formula II-a.

27. A composition comprising a compound of formula I-b1: 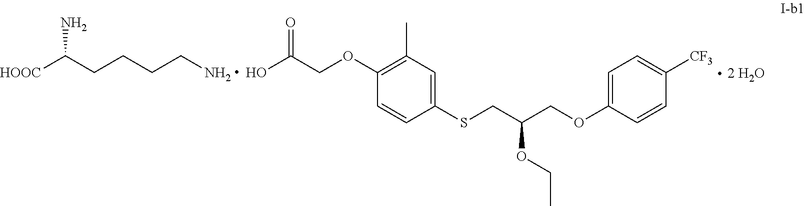 [SMILES: NCCCC[C@@H](N)C(=O)O ; CCO[C@H](COc1ccc(C(F)(F)F)cc1)CSc1ccc(OCC(=O)O)c(C)c1]
wherein the composition comprises at least about 97% w/w of the compound of formula I-b1, and less than about 2% w/w of a compound selected from: 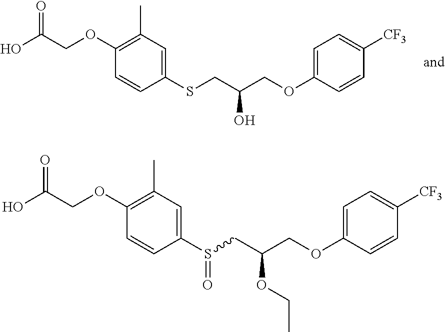 [SMILES: Cc1cc(SC[C@H](O)COc2ccc(C(F)(F)F)cc2)ccc1OCC(=O)O ; CCO[C@H](COc1ccc(C(F)(F)F)cc1)CS(=O)c1ccc(OCC(=O)O)c(C)c1]
or a combination thereof.
